SEQUENCE LISTING (18 sequences)

SEQ ID NO: 1 - protein, 42 aa, Artificial Sequence
  41BBKR
RRGRRRLLYIFRQPFMRPVQTTQEEDGCSCRFPEEEEGGCEL

SEQ ID NO: 2 - protein, 41 aa, Artificial Sequence
  CD28KR
RSRRSRLLHSDYMNMTPRRPGPTRRHYQPYAPPRDFAAYRS

SEQ ID NO: 3 - protein, 113 aa, Artificial Sequence
  CD3KR
RVRFSRSADAPAYQQGQNQLYNELNLGRREEYDVLDRRRGRDPEMGGRPQRRRNPQEGLYNELQRDRMAEAYSEIGMRGERRRGRGHDGLYQGLSTATRDTYDALHMQALPPR

SEQ ID NO: 4 - protein, 155 aa, Artificial Sequence
  41BB-CD3KR
RRGRRRLLYIFRQPFMRPVQTTQEEDGCSCRFPEEEEGGCELRVRFSRSADAPAYQQGQNQLYNELNLGRREEYDVLDRRRGRDPEMGGRPQRRRNPQEGLYNELQRDRMAEAYSEIGMRGERRRGRGHDGLYQGLSTATRDTYDALHMQALPPR

SEQ ID NO: 5 - protein, 154 aa, Artificial Sequence
  CD28-CD3KR
RSRRSRLLHSDYMNMTPRRPGPTRRHYQPYAPPRDFAAYRSRVRFSRSADAPAYQQGQNQLYNELNLGRREEYDVLDRRRGRDPEMGGRPQRRRNPQEGLYNELQRDRMAEAYSEIGMRGERRRGRGHDGLYQGLSTATRDTYDALHMQALPPR

SEQ ID NO: 6 - protein, 242 aa, Artificial Sequence
  anti-CD19 (FMC63)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 7 - protein, 241 aa, Artificial Sequence
  anti-GD2 (14g2a)
DVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKGGGGSGGGGSGGGGSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSS

SEQ ID NO: 8 - protein, 21 aa, Artificial Sequence
  CD8 signal peptide
MALPVTALLLPLALLLHAARP

SEQ ID NO: 9 - protein, 45 aa, Artificial Sequence
  CD8 hinge region
TTTPAPRPPTPAPTIASQPLSLRPEACRPAAGGAVHTRGLDFACD

SEQ ID NO: 10 - protein, 24 aa, Artificial Sequence
  CD8 transmembrane region
IYIWAPLAGTCGVLLLSLVITLYC

SEQ ID NO: 11 - protein, 497 aa, Artificial Sequence
  CD19 41BB KR CAR
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRGRRRLLYIFRQPFMRPVQTTQEEDGCSCRFPEEEEGGCELRVRFSRSADAPAYQQGQNQLYNELNLGRREEYDVLDRRRGRDPEMGGRPQRRRNPQEGLYNELQRDRMAEAYSEIGMRGERRRGRGHDGLYQGLSTATRDTYDALHMQALPPR

SEQ ID NO: 12 - protein, 496 aa, Artificial Sequence
  CD19 CD28 KR CAR
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRSRRSRLLHSDYMNMTPRRPGPTRRHYQPYAPPRDFAAYRSRVRFSRSADAPAYQQGQNQLYNELNLGRREEYDVLDRRRGRDPEMGGRPQRRRNPQEGLYNELQRDRMAEAYSEIGMRGERRRGRGHDGLYQGLSTATRDTYDALHMQALPPR

SEQ ID NO: 13 - protein, 496 aa, Artificial Sequence
  GD2 41BB KR CAR
MALPVTALLLPLALLLHAARPEQKLISEEDLDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKGGGGSGGGGSGGGGSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSTTTPAPRPPTPAPTIASQPLSLRPEACRPAAGGAVHTRGLDFACDIYIWAPLAGTCGVLLLSLVITLYCRRGRRRLLYIFRQPFMRPVQTTQEEDGCSCRFPEEEEGGCELRVRFSRSADAPAYQQGQNQLYNELNLGRREEYDVLDRRRGRDPEMGGRPQRRRNPQEGLYNELQRDRMAEAYSEIGMRGERRRGRGHDGLYQGLSTATRDTYDALHMQALPPR

SEQ ID NO: 14 - protein, 495 aa, Artificial Sequence
  GD2 CD28 KR CAR
MALPVTALLLPLALLLHAARPEQKLISEEDLDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKGGGGSGGGGSGGGGSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSTTTPAPRPPTPAPTIASQPLSLRPEACRPAAGGAVHTRGLDFACDIYIWAPLAGTCGVLLLSLVITLYCRSRRSRLLHSDYMNMTPRRPGPTRRHYQPYAPPRDFAAYRSRVRFSRSADAPAYQQGQNQLYNELNLGRREEYDVLDRRRGRDPEMGGRPQRRRNPQEGLYNELQRDRMAEAYSEIGMRGERRRGRGHDGLYQGLSTATRDTYDALHMQALPPR

SEQ ID NO: 15 - DNA, 1491 bp, Artificial Sequence
  CD19 41BB KR CAR
atggccttaccagtgaccgccttgctcctgccgctggccttgctgctccacgccgccaggccggagcagaagctgatcagcgaggaggacctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccaccactcccgcaccccgccctcctactcctgcccctaccattgctagccaaccgcttagtctgagacctgaggcctgtaggcccgctgctggtggcgctgtgcacacccgaggattggacttcgcttgcgacatctacatctgggcacctctggctgggacctgcggcgtgttgttgttgagcctggtgattacgctgtactgtagacggggcagacgcagactcctgtatatattccgccaaccatttatgagaccagtacaaactactcaagaggaagatggctgtagctgccgatttccagaagaagaagaaggaggatgtgaactgagagtgagattcagcaggagcgcagacgcccccgcgtaccagcagggccagaaccagctctataacgagctcaatctaggacgaagagaggagtacgatgttttggaccgcagacgtggccgggaccctgagatggggggaagaccgcagagaaggcgcaaccctcaggaaggcctgtacaatgaactgcagcgcgatagaatggcggaggcctacagtgagattgggatgagaggcgagcgccggaggggcagagggcacgatggcctttaccagggtctcagtacagccacccgcgacacctacgacgcccttcacatgcaggccctgcctcctcgc

SEQ ID NO: 16 - DNA, 1488 bp, Artificial Sequence
  CD19 CD28 KR CAR
atggccttaccagtgaccgccttgctcctgccgctggccttgctgctccacgccgccaggccggagcagaagctgatcagcgaggaggacctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccaccactcccgcaccccgccctcctactcctgcccctaccattgctagccaaccgcttagtctgagacctgaggcctgtaggcccgctgctggtggcgctgtgcacacccgaggattggacttcgcttgcgacatctacatctgggcacctctggctgggacctgcggcgtgttgttgttgagcctggtgattacgctgtactgtaggagtagaaggagcaggctcctgcacagtgactacatgaacatgactccccgccgccccgggcccacccgcagacattaccagccctatgccccaccacgcgacttcgcagcctatcgctccagagtgagattcagcaggagcgcagacgcccccgcgtaccagcagggccagaaccagctctataacgagctcaatctaggacgaagagaggagtacgatgttttggaccgcagacgtggccgggaccctgagatggggggaagaccgcagagaaggcgcaaccctcaggaaggcctgtacaatgaactgcagcgcgatagaatggcggaggcctacagtgagattgggatgagaggcgagcgccggaggggcagagggcacgatggcctttaccagggtctcagtacagccacccgcgacacctacgacgcccttcacatgcaggccctgcctcctcgc

SEQ ID NO: 17 - DNA, 1488 bp, Artificial Sequence
  GD2 41BB KR CAR
atggccttaccagtgaccgccttgctcctgccgctggccttgctgctccacgccgccaggccggagcagaagctgatcagcgaggaggacctggatgttgtcatgactcaaacccctttatctttgcccgtatcccttggtgaccaggcttcaatttcgtgtcgtagtagccaatctctcgtgcatcgcaatggcaacacatatctacactggtacctgcagaaaccaggacaatccccgaagttattgatccataaagtttcaaatcgattttcgggggtccctgatcggttcagtggtagcggctctggaacggactttactcttaagatatccagagtagaagccgaggatctcggggtgtatttctgctcacagtcgacccacgttcccccactaacatttggtgcaggcacgaaactggaattaaagggtggcggtggctcgggcggtggtgggtcgggtggcggcggatctgaagttcaattattgcagtctggtcctgagcttgaaaaacccggcgcttccgtcatgatttcatgtaaggcctcgggaagtagctttactgggtataatatgaactgggtacgtcaaaatatcggtaaatctctcgaatggataggcgcaattgatccatactatggagggacctcctacaaccagaagttcaaaggtcgcgcgacactaacggtggacaagtcatcgagtactgcttatatgcatctgaaaagcttaacctctgaagattccgccgtttactattgcgtctcaggcatggagtactggggacaagggacatcggtaacggtgagtagcaccaccactcccgcaccccgccctcctactcctgcccctaccattgctagccaaccgcttagtctgagacctgaggcctgtaggcccgctgctggtggcgctgtgcacacccgaggattggacttcgcttgcgacatctacatctgggcacctctggctgggacctgcggcgtgttgttgttgagcctggtgattacgctgtactgtagacggggcagacgcagactcctgtatatattccgccaaccatttatgagaccagtacaaactactcaagaggaagatggctgtagctgccgatttccagaagaagaagaaggaggatgtgaactgagagtgagattcagcaggagcgcagacgcccccgcgtaccagcagggccagaaccagctctataacgagctcaatctaggacgaagagaggagtacgatgttttggaccgcagacgtggccgggaccctgagatggggggaagaccgcagagaaggcgcaaccctcaggaaggcctgtacaatgaactgcagcgcgatagaatggcggaggcctacagtgagattgggatgagaggcgagcgccggaggggcagagggcacgatggcctttaccagggtctcagtacagccacccgcgacacctacgacgcccttcacatgcaggccctgcctcctcgc

SEQ ID NO: 18 - DNA, 1485 bp, Artificial Sequence
  GD2 CD28 KR CAR
atggccttaccagtgaccgccttgctcctgccgctggccttgctgctccacgccgccaggccggagcagaagctgatcagcgaggaggacctggatgttgtcatgactcaaacccctttatctttgcccgtatcccttggtgaccaggcttcaatttcgtgtcgtagtagccaatctctcgtgcatcgcaatggcaacacatatctacactggtacctgcagaaaccaggacaatccccgaagttattgatccataaagtttcaaatcgattttcgggggtccctgatcggttcagtggtagcggctctggaacggactttactcttaagatatccagagtagaagccgaggatctcggggtgtatttctgctcacagtcgacccacgttcccccactaacatttggtgcaggcacgaaactggaattaaagggtggcggtggctcgggcggtggtgggtcgggtggcggcggatctgaagttcaattattgcagtctggtcctgagcttgaaaaacccggcgcttccgtcatgatttcatgtaaggcctcgggaagtagctttactgggtataatatgaactgggtacgtcaaaatatcggtaaatctctcgaatggataggcgcaattgatccatactatggagggacctcctacaaccagaagttcaaaggtcgcgcgacactaacggtggacaagtcatcgagtactgcttatatgcatctgaaaagcttaacctctgaagattccgccgtttactattgcgtctcaggcatggagtactggggacaagggacatcggtaacggtgagtagcaccaccactcccgcaccccgccctcctactcctgcccctaccattgctagccaaccgcttagtctgagacctgaggcctgtaggcccgctgctggtggcgctgtgcacacccgaggattggacttcgcttgcgacatctacatctgggcacctctggctgggacctgcggcgtgttgttgttgagcctggtgattacgctgtactgtaggagtagaaggagcaggctcctgcacagtgactacatgaacatgactccccgccgccccgggcccacccgcagacattaccagccctatgccccaccacgcgacttcgcagcctatcgctccagagtgagattcagcaggagcgcagacgcccccgcgtaccagcagggccagaaccagctctataacgagctcaatctaggacgaagagaggagtacgatgttttggaccgcagacgtggccgggaccctgagatggggggaagaccgcagagaaggcgcaaccctcaggaaggcctgtacaatgaactgcagcgcgatagaatggcggaggcctacagtgagattgggatgagaggcgagcgccggaggggcagagggcacgatggcctttaccagggtctcagtacagccacccgcgacacctacgacgcccttcacatgcaggccctgcctcctcgc

(19) United States
(12) Patent Application Publication
(10) Pub. No.: US 2022/0280566 A1
(43) Pub. Date: Sep. 8, 2022

(54) UBIQUITINATION-LACKING CHIMERIC ANTIGEN RECEPTOR AND USE THEREOF

(71) Applicant: ShanghaiTech University, Shanghai (CN)

(72) Inventors: Haopeng WANG, Shanghai (CN); Wentao LI, Shanghai (CN); Shizhen QIU, Shanghai (CN); Ping WEI, Shanghai (CN)

(73) Assignee: ShanghaiTech University, Shanghai (CN)

(21) Appl. No.: 17/603,937

(22) PCT Filed: Oct. 31, 2019
(86) PCT No.: PCT/CN2019/114655

§ 371 Date: May 12, 2022

(30) Foreign Application Priority Data
Apr. 16, 2019 (CN) 201910302508.X

(51) Int. Cl. A61K 35/17; C07K 14/725; C07K 14/705; A61P 35/00; C12N 15/62; C12N 5/0783; C12N 7/00; C12N 15/86

(52) U.S. Cl. CPC A61K 35/17; C07K 14/7051; C07K 14/70521; C07K 14/70578; A61P 35/00; C12N 15/625; C12N 5/0636; C12N 7/00; C12N 15/86; A61K 38/00

(57) ABSTRACT

The present invention provides a chimeric antigen receptor, which includes: an extracellular domain, a transmembrane domain, and an intracellular domain connected in sequence. The extracellular domain includes an antigen recognition region; the intracellular domain includes a costimulatory signaling region and a CD3ζ intracellular region that are connected in sequence, to form a costimulatory signaling region-CD3ζ intracellular region; and the costimulatory signaling region-CD3ζ intracellular region is a polypeptide formed by mutation of lysine in a wild-type costimulatory signaling region-CD3ζ intracellular region into arginine. The present invention provides a method for optimization and modification of CAR-T, in which all lysine sites in an intracellular segment of CAR are mutated into arginine, thereby blocking ubiquitination modification of the CAR after antigen challenge. This strategy is applicable to different CARs and changing different intracellular costimulatory domains, and in particular provides a solution to the problem of poor proliferation of CAR-T in solid tumors.

### CROSS REFERENCE TO RELATED APPLICATION

The present application claims the benefit of priority to Chinese Patent Application No. CN 201910302508.X. entitled “UBIQUITINATION-LACKING CHIMERIC ANTIGEN RECEPTOR AND USE THEREOF”, filed with CNIPA on Apr. 16, 2019, the disclosure of which is incorporated herein by reference in its entirety for all purposes.

### FIELD OF TECHNOLOGY

The present invention relates to the field of chimeric antigen receptors, and specifically to a ubiquitination-lacking chimeric antigen receptor and use thereof.

### BACKGROUND

Chimeric antigen receptors are abbreviated as CAR, and T cells carrying CAR for specific tumor antigen are CAR-T cells. As a rising star of adoptive cell therapy. CAR-T therapy has achieved significant success in the treatment of a variety of tumors, particularly in the treatment of hematological tumors. In 2017, the U.S. FDA successively approved two commercialized CAR-T products targeting the CD19 antigen for treating malignant leukemia and lymphoma with remarkable curative effect However, there are still many limitations of CAR-T therapy in the treatment of solid tumors, and the inability of CAR-T cells to expand efficiently and consistently in patients due to a number of factors is considered to be 3 major manifestation of limiting the CAR-T anti-tumor activity. Studies have demonstrated that enhancing the proliferative capacity of CAR-T cells in vivo can significantly improve the efficacy of CAR-T therapy, both in hematologic tumor therapy and solid tumor therapy, which provides a direction to optimize CAR-T design. Existing optimization design strategies mainly include co-expression of cytokines, such as IL-7, IL-15, IL-18 and the like (13-15), that promote T cell sternness and proliferative capacity in CAR-T cells. However, such design often limits the large-scale industrial production and clinical practice due to the factors such as large construction difficulty, low conversion efficiency and the like. Designing a modification to the CAR structure itself is a more optimal solution.

### SUMMARY

In view of the above-mentioned drawbacks of the prior art, an objective of the present invention is to provide a ubiquitination-lacking chimeric antigen receptor and use thereof.

In order to accomplish the above and other related objectives, a first aspect of the present invention provides a chimeric antigen receptor, which includes

an extracellular domain, a transmembrane domain, and an intracellular domain connected in sequence,

the extracellular domain includes an antigen recognition region:

the intracellular domain includes a costimulatory signaling region and a CD3ζ intracellular region that are connected in sequence to form a costimulatory signaling region-CD3ζ intracellular region; and

the costimulatory signaling region-CD3ζ intracellular region is a polypeptide formed by mutation of lysine in a wild-type costimulatory signaling region-CD3ζ intracellular region into arginine.

A second aspect of the present invention provides a polynucleotide sequence, which is selected from:

(1) a polynucleotide sequence encoding the aforementioned chimeric antigen receptor: and

(2) a complementary sequence of the polynucleotide sequence as described in (1).

A third aspect of the present invention provides a nucleic acid construct, which includes the aforementioned polynucleotide sequence

Preferably, the nucleic acid construct is a vector.

More preferably, the nucleic acid construct is a lentiviral vector containing a replication initiation site. 3′LTR. 5′LTR and the aforementioned polynucleotide sequence.

A fourth aspect of the present invention provides a lentivirus, which includes the aforementioned nucleic add construct.

A fifth aspect of the present invention provides a method for activating T cells in vitro, which includes the step of infecting the T cells with the aforementioned lentivirus.

A sixth aspect of the present invention provides a genetically modified T cell or a pharmaceutical composition containing the genetically modified T cell, the cell contains the aforementioned polynucleotide sequence, or contains the aforementioned nucleic acid construct, or is infected with the aforementioned lentivirus, or is prepared by the aforementioned method

A seventh aspect of the present invention provides the use of the aforementioned chimeric antigen receptor, the aforementioned polynucleotide sequence, the aforementioned nucleic acid construct, or the aforementioned lentivirus in preparing activated T cells and/or inhibiting T cell degradation

An eighth aspect of the present invention provides the use of the aforementioned chimeric antigen receptor, the aforementioned polynucleotide sequence, the aforementioned nucleic add construct, the aforementioned lentivirus or the aforementioned genetically modified T cell in any one or more of the following applications: (1) preparing tumor therapeutic drugs; (2) improving tumor killing efficiency; (3) maintaining T cell proliferation capacity; and (4) inhibiting tumor development;

preferably, the tumor is selected from one or more of leukemia or lymphoma, and more preferably, the tumor is selected from B-cell lymphoma, mantle cell lymphoma, acute lymphocytic leukemia, chronic lymphocytic leukemia, hairy cell leukemia, and acute myeloid leukemia.

A ninth aspect of the present invention provides a cell therapy in which T cells are genetically modified to express the aforementioned CAR, and CAR-T cells are injected into subjects in need thereof.

As mentioned above, the ubiquitination-lacking chimeric antigen receptors and use thereof of the present invention have the following beneficial effects.

The present invention mutates all lysine sites in an intracellular segment of the CAR into arginine, thereby blocking the ubiquitination modification of the CAR after antigen challenge. This strategy is applicable to different CARs and changing different intracellular costimulatory domains.

On the other hand, this modification method is applied to the CAR-T with 41BB as the intracellular costimulatory domain, compared with the traditional 41BB CAR-T, this KR-mutated CAR-T can better respond to proliferation challenged by target cells during in-vitro culture, and has a phenotype that is biased toward central memory T cell differentiation, in addition, it shows stronger killing activity in a long-term in-vitro tumor killing test This enhanced phenotype benefits from the fact that 41BB KR CAR-T is more dependent on oxidative phosphorylation in metabolism and has stronger mitochondrial production capacity and function. Moreover, the modified 4IBB KR CAR-T can produce more antioxidant protein products than the traditional 41BB WT CAR-T. which enhances the survival and proliferation capacity of cells.

In another aspect, the present invention compares the anti-tumor effects in vivo of 41BB KR CAR-T and 4IBB WT CAR-T in tumor mouse models In terms of proliferation level, 41BB KR CAR-T has stronger proliferation response and the proliferation capacity is more durable. In terms of cell differentiation phenotype, whether in the spleen, blood, or tumor, the modified CAR-T has accumulated more central memory T cells and reduced the differentiation to terminal effector T cells. Therefore, the modified CAR-T can more effectively infiltrate the tumor tissue for killing. Under the same T cell injection dose, the modified CAR-T can more effectively control the development of the tumor

The present invention provides a method for optimization and modification of CAR-T, and particularly provides a solution to the problem of poor proliferation capacity of CAR-T in solid tumors.

### BRIEF DESCRIPTION OF THE DRAWINGS

FIG. 1A is a graph showing results of interaction of Jurkat cells expressing CD19-ζ CAR with K562 cells expressing CD19 specific antigen or Meso non-antigen

FIG. 1B is a graph showing results of interaction of Jurkat cells expressing CD19-ζ, CD19-CD28ζ, and CD19-BBζ respectively with target cells.

FIG. 2 is a graph showing results of down-regulation and degradation inhibited by KR-mutated CAR. (FIG. 2A compares ubiquitin modification after expressing CD19-41BBζ WT CAR or KR CAR in human primary T cells. FIG. 2B compares the response of CD19-41BBζ WT CAR or KR CAR to target cell challenge after being expressed in human primary T cells. FIG. 2C and FIG. 2D compare the effect of KR mutation on down-regulation of CD19-CD28ζ and GD2-CD28ζ CAR in human primary T cells FIG. 2E shows the effect of KR mutation on degradation of CD19-41BBζ CAR in human primary T cells. FIG. 2F is a quantitative result of FIG. 2E, and KR mutation effectively inhibits the degradation of CAR in this reaction. FIG. 2G shows the effect of KR mutation on degradation of GD2-41BBζ and GD2-CD28ζ CAR in Jurkat cells. FIG. 2H is a quantitative result of FIG. 2G, and KR mutation effectively inhibits the degradation of CAR mediated by antigen challenge.)

FIG. 3 is a graph showing the effect of KR mutation on CAR-T in-vitro function (All of the in-vitro function experiments are performed on human primary T cells. FIG. 3A shows the effect of KR mutation on proliferation of CAR-T after responding to target cell challenge FIG. 3B shows differentiation of WT and KR CAR-T under in-vitro culture conditions, and FACS analysis is performed with CD62L and CD45RA as differentiation indicators. FIG. 3C is a statistical analysis of FIG. 3B, and KR mutation allows CAR-T to differentiate more into central memory T cells and reduces the differentiation into terminal effector T cells. FIG. 3D is a differentiation phenotype of CAR-T after being challenged by target cells for 4 days, CAR-T and irradiated target cells are co-incubated at a ratio of 2:1, and FACS analysis is performed with CD27 and CD45RO as differentiation indicators FIG. 3E is a statistical analysis of FIG. 3D, and KR mutation allows CAR-T to form more central memory T cells after response to target cell challenge. FIG. 3F shows an in-vitro killing activity test of CAR-T.

FIG. 4 is a graph revealing a molecular basis of CD19-41BBζ KR CAR which can accumulate more central memory T cells and enhanced proliferation phenotypes. (This part of experiment is performed and verified on human primary T cells. FIG. 4A uses a Seahorse machine to detect the difference in oxidative metabolism between WT CAR-T and KR CAR-T, and the tested cells are first challenged with target cells for two weeks. FIG. 4B is a statistical analysis graph of main parameters of FIG. 4A. FIG. 4C and FIG. 4D verify that KR CAR-T has stronger mitochondrial production capacity through the FACS and fluorescence confocal detection methods, respectively, with mitotracker being a detection marker. FIG. 4E uses fluorescence quantitative PCR to detect the changes of four important genes related to mitochondrial production and function in WT and KR CAR-T before and after being challenged with irradiated target cells. FIG. 4F and FIG. 4G verify that KR CAR-T can produce more anti-apoptotic factor Bcl-xL after being challenged by target cells through westernblot and FACS detection methods, respectively. FIG. 4H uses fluorescence quantitative PCR to verify that KR CAR-T produces more mRNA of 41BB downstream anti-apoptotic products Bcl-xL and Bf11 after being challenged by target cells, compared with WT CAR-T.)

FIG. 5 is a graph comparing in-vivo functions of CD19-42BBζ WT and KR CAR-T in tumor mouse models (FIG. 5A compares in vivo down-regulation of WT and KR CAR FIG. 5B shows the effect of WT and KR CAR-T on tumor infiltrating. FIG. 5C shows changes in number of WT and KR CAR-T in mouse spleens 10 days, 20 days, and 40 days after injection of CAR-T cells into tumor-bearing mice FIG. 5D shows the comparison of CAR-T differentiation phenotypes in spleens of tumor-bearing mice. FIG. 5E is a statistical analysis of FIG. 5D. FIG. 5F shows the comparison of CAR-T differentiation phenotypesin peripheral bloods of tumor-bearing mice. FIG. 5G is a statistical analysis graph of FIG. 5F. FIG. 5H shows the comparison of CAR-T differentiation phenotypes in tumor tissues. FIG. 5I is a statistical analysis graph of FIG. 5H.)

FIG. 6 is a graph comparing the anti-tumor effects of CD19-41BBζ WT and KR CAR-T. (FIG. 6A shows development of target cells expressing firefly luciferase genes in NSG mice injected with different T cells FIG. 6B is a graph showing quantitative results of 6A.)

FIG. 7 is a graph comparing the effect of KR mutation on phenotype of CD19-41BBζ and phenotype of CD19-CD28ζ CAR-T cells. (FIG. 7A and FIG. 7B compare expression levels of CD69 and ICOS on Juikat cells after expressing CD19-41BBζ or CD19-CD28ζ and a corresponding mutant CAR thereof, respectively FIG. 7C shows an expression level of PD-1 in human primary T cells expressing CD19-41BBζ or CD19-CD28ζ and a corresponding mutant CAR The relative expression level of PD-1 is shown with the expression level of CD19-41BBζ WT CAR-T cells as a reference FIG. 7D to FIG. 7G show differentiation phenotype results of primary T cells after expressing CAR. FIG. 7D and FIG. 7E show T cells expressing CD19 CAR FIG. 7D shows CD4 T cells and FIG. 7E shows CD8 T cells. FIG. 7F and FIG. 7G show T cells expressing GD2 CAR FIG. 7F shows CD4 T cells and FIG. 7G shows CD8 T cells.)

FIG. 8 is a graph comparing response effects of CD19-41BBζ WT and KR CAR-T cells on a tumor cell rechallenge model. (FIG. 8A shows that mice are infused with 1.5×10* CAR-T cells for treatment 4 days after being subcutaneously inoculated with 2×10⁶ tumor cells. When no tumor signal is detected in all mice, tumor cells were re-inoculated: infusing with 1×10⁶ tumor cells via tail vein, the arrow represents the time point of rechallenge, and each line represents a mouse FIG. SB compares in-vivo anti-tumor effects of two kinds of CAR-T cells 10 days after being challenged by target cells. 4 days after subcutaneously inoculating 1×10⁶ tumor cells. 2×10⁶ T cells are injected via the tail vein, with 6 mice in each group. FIG. 8C compares in-vivo anti-tumor effects of two kinds of CAR-T cells 21 days after being challenged by target cells. 4 days after subcutaneously inoculating 3×10⁶ tumor cells. 1×10⁶ T cells are injected via the tail vein, with 4 to 6 mice in each group.)

### DETAILED DESCRIPTION

The chimeric antigen receptor of the present invention includes:

an extracellular domain, a transmembrane domain, and an intracellular domain connected in sequence

the extracellular domain includes an antigen recognition region;

the intracellular domain includes a costimulatory signaling region and a CD3ζ intracellular region that are connected in sequence to form a costimulatory signaling region-CD3ζ intracellular region, and

the costimulatory signaling region-CD3ζ intracellular region is a polypeptide formed by mutation of lysine in a wild-type costimulatory signaling region-CD3ζ intracellular region into arginine.

In one embodiment, the costimulatory signaling region is selected from an intracellular region of CD27, CD28, CD134, 41BB or ICOS.

Preferably, the costimulatory signaling region is selected from 41BB intracellular region or CD28 intracellular region.

In one embodiment, the amino acid sequence of the 41BB intracellular region is shown in SEQ ID NO: 1 (41BBKR) Specifically:

RRGRRRLLYIFRQPFMRPVQTTQEEDGCSCRFPEEEEGGCEL.

In one embodiment, the amino acid sequence of the CD28 intracellular region is shown in SEQ ID NO: 2 (CD28KR). Specifically:

RSRRSRLLLHSDYMNMTPRRPGPTRRHYQPYAPPRDFAAYRS.

In one embodiment, the amino add sequence of the CD3ζ intracellular region is shown in SEQ ID NO: 3 (CD3KR). Specifically:

RVRFSRSADAPAYQQGQNQLYNELNLGRREEYDVLDRRRGRDP
EMGGRPQRRRNPQEGLYNELQRDRMAEAYSEIGMRGERRRGRG
HDGLYQGLSTATRDTYDALHMQALPPR

In one embodiment, the amino add sequence of the costimulatory signaling region-CD3ζ intracellular region is shown in SEQ ID NO: 4(41BB-CD3KR) or SEQ ID NO: 5 (CD28-CD3KR). Specifically:

(SEQ ID NO: 4)
RRGRRRLLYIFRQPFMRPVQTTQEEDGCSCRFPEEEEGGCEL
RVRFSRSADAPAYQQGQNQLYNELNLGRREEYDVLDRRRGRD
PEMGGRPQRRRNPQEGLYNELQRDRMAEAYSEIGMRGERRRG
RGHDGLYQGLSTATRDTYDALHMQALPPR.
(SEQ ID NO: 5)
RSRRSRLLHSDYMNMIPRRPGPTRRHYQPYAPPRDFAAYRSR
VRFSRSADAPAYQQGQNQLYNELNLGRREEYDVLDRRRGRDP
EMGGRPQRRRNPQEGLYNELQRDRMAEAYSEIGMRGERRRGR
GHDGLYQGLSTATRDTYDALHMQALPPR

In one embodiment, the antigen recognition region is selected from a single chain antibody against a tumor surface antigen, and the tumor surface antigen is selected from one or more of CD19, CD123, CD30, BCMA, Her2, IL13Rα2 and GD2.

Preferably, the tumor surface antigen is selected from CD19 or GD2.

In one embodiment, the single chain antibody contains a light chain variable region and a heavy chain variable region.

In one embodiment, the light chain variable region and the heavy chain variable region are connected by a linker sequence.

In one embodiment, the single chain antibody is selected from FMC63 or GD2.

In one embodiment, the single chain antibody contains a light chain variable region and a heavy chain variable region of a monodonal antibody FMC63, and the light chain variable region and the heavy chain variable region arc optionally connected by a linker.

In one embodiment, the single chain antibody contains a light chain variable region and a heavy chain variable region of a monoclonal antibody 14g2a, and the light chain variable region and the heavy chain variable region are optionally connected by a linker.

Further, the extracellular domain further includes a signal peptide and or a hinge region to form a signal peptide-antigen recognition region and hinge region connected in sequence.

Preferably, the signal peptide is selected from a CD8α signal peptide and or the hinge region is selected from a CDSα hinge region.

In one embodiment, the amino acid sequence of the antigen recognition region is shown in SEQ ID NO: 6 or SEQ ID NO: 7.

Specifically:

(SEQ ID NO: 6, anti-CD19 (FMC63))
DIQMTQTTSSLSASLGDRVTISCRASQDISKYLNW
YQQKPDGTVKLLIYHTSRLHSGVPSRFSGSGSGTD
YSLUSNLEQEDIATYFCQQGNTLPYTFGGGTKLEI
TGGGGSGGGGSGGGGSEVKLQESGPGLVAPSQSLS
VTCTVSGVSLPDYGVSWIRQPPRKGLEWLGVIWGS
ETTYYNSALKSRLTIIKDNSKSQVFLKMNSLQTDD
TAIYYCAKHYYYGGSYAMDYWGQGTSVTVSS.
(SEQ ID NO: 7, anti-GD2(14g2a))
DVVMTQTPLSLPVSLGDQASISCRSSQSLVHRNGN
TYLHWYLQKPGQSPKLLIHKVSNRFSGVTDRFSGS
GSGTDFTLKISRVEAEDLGVYFCSQSTHVPPLTFG
AGTKLELKGGGGSGGGGSGGGGSEVQLLQSGPELE
KPGASVMISCKASGSSFTGYNMNWVRQNIGKSLEW
IGAIDPYYGGTSYNQKFKGRATLTVDKSSSTAYMH
LKSLTSEDSAVYYCVSGMEYWGQGTSVTVSS.

In one embodiment, the amino acid sequence of the CD8α signal peptide is shown in SEQ ID NO: Specifically:

MALPVTALLLPLALLLHAARP.

In one embodiment, the amino acid sequence of the CD8α hinge region is shown in SEQ ID NO: 9. Specifically:

TTTPAPRPPTPAPTIASQPLSLRPEACRP
AAGGAVHTRGLDFACD.

The transmembrane domain is selected from a transmembrane region of CD4, CD8α, OX40 or H2-Kb. Preferably, it is selected from the transmembrane region of CD8α.

In one embodiment, the amino acid sequence of the CD8α transmembrane region is shown in SEQ ID NO: 10. Specifically.

IYTWAPLAGTCGVLLLSLVITLYC.

In one embodiment, the amino acid sequence of the chimeric antigen receptor is shown in SEQ ID NO: 11-14. Specifically:

(SEQ ID NO: 11, CD19 41BB KR CAR)
MALPVTALLLPLALLLHAARPEQKLISEEDLDIQM
TQTTSSLSASLGDRVTISCRASQDISKYLNWYQQK
PDGTVKLLIYHTSRLHSGYTSRFSGSGSGTDYSLT
ISNLEQEDLATYFCQQGNTLPYTFGGGTKLEFIGG
GGSGGGGSGGGGSEVKLQESGPGLVAPSQSLSVTC
TVSGVSLPDYGVSWIRQPPRKGLEWLGVIWGSETT
YYNSALKSRLTIIKDNSKSQVFLKMNSLQTDDTAI
YYCAKHYYYGGSYAMDYWGQGTSVTVSSTTTPAPR
PPTPAPTIASQPLSLRPEACRPAAGGAVHTRGLDF
ACDIYIWAPLAGTCGVLLLSLMTLYCRRGRRRLLY
IFRQPEMRPVQTIQEEDGCSCRFPEEEEGGCELER
VRFSRSADAPAYQQGQNQLYNELNLGRREEYDVLD
RRRGRDPEMGGRPQRRRNPQEGLYNELQRDRMAEA
YSEIGMRGERRRGRGHDGLYQGLSTATRDTYDALH
MQALPPR.
(SEQ ID NO: 12, CD19 CD28 KR CAR)
MALPVTALLLPLALLLHAARPEQKLISEEDLDIQM
TQTTSSLSASLGDRVTISCRASQDISKYLNWYQQK
PDGTVKLHYHTSRLHSGVPSRFSGSGSGTDYSLTI
SNLEQEDIATYFCQQGNTLPYTFGGGTKLEITGGG
GSGGGGSGGGGSEVKLQESGPGLVAPSQSLSVTCT
VSGVSLPDYGVSWIRQPPRKGLEWLGVIWGSETTY
YNSALKSRLTIIKDNSKSQVFLKMNSLQTDDTAIY
YCAKHYYYGGSYAMDYWGQGTSVTVSSTTTPAPRP
FTPAPTIASQPLSLRPEACRPAAGGAVHTRGLDFA
CDIYIWAPLAGTCGVLLLSLYITLYCRSRRSRHHS
DYMNMTPRRPGPTRRHYQPYAPPRDFAAYRSRVRF
SRSADAPAYQQGQNQLYNELNLGRREEYDVLDRRR
GRDPEMGGRPQRRRNPQEGLYNELQRDRMAEAYSE
IGMRGERRRGRGHDGLYQGLSTATRDTYDALHMQA
LPPR.
(SEQ ID NO: 13, GD2 41BB KR CAR)
MALPVTALLLPLALLLHAARPEQKLISEEDLDVVM
TQTPLSLPVSLGDQASISCRSSQSLVHRNGNTYLH
WYLQKPGQSPKLLIHKVSNRFSGVPDRFSGSGSGT
DFTLKISRVEAEDLGVYFCSQSTHVPPLTFGAGTK
LELKGGGGSGGGGSGGGGSEVQLLQSGPELEKPGA
SVMISCKASGSSFTGYNMNWVRQNIGKSLEWIGAI
DPYYGGTSYNQKFKGRATLTVDKSSSTAYMHLKSL
TSEDSAVYYCVSGMEYWGQGTSVTVSSTTTPAPRP
PTPAPTIASQPLSLRPEACRPAAGGAVHTRGLDFA
CDIYIWAPLAGTCGVLLLSLVITLYCRRGRRRLLY
IFRQPFMRPVQTTQEEDGCSCRFPEEEEGGCELRV
RFSRSADAPAYQQGQNQLYNELNLGRREEYDVLDR
RRGRDPEMGGRPQRRRNPQEGLYNELQRDRMAEAY
SEIGMRGERRRGRGHDGLYQGLSTATRDTYDALHM
QALPPR.
(SEQ ID NO: 14, GD2 CD28 KR CAR)
MALPVTALLLPLALLLHAARPEQKLISEEDLDVVM
TQTPLSLPVSLGDQASISCRSSQSLVHRNGNTYLH
WYLQKPGQSPKLLIHKVSNRFSGVPDRFSGSGSGT
DFTLKISRVEAEDLGVYFCSQSTHVPPLTFGAGTK
LELKGGGGSGGGGSGGGGSEVQLLQSGPELEKPGA
SVMISCKASGSSFTGYNMNWVRQNTGKSLEWIGAI
DPYYGGTSYNQKFKGRATLTVDKSSSTAYMHLKSL
TSEDSAVYYCVSGMEYWGQGTSVTVSSTTTPAPRP
PTPAPTIASQPLSLRPEACRPAAGGAVHTRGLDFA
CDIYIWAPLAGTCGVLLLSLVTILYCRSRRSRLLH
SDYMNMTPRRPGPTRRHYQPYAPPRDFAAYRSRVR
FSRSADAPAYQQGQNQLYNELNLGRREEYDVLDRR
RGRDPEMGGRPQRRRNPQEGLYNELQRDRMAEAYS
EIGMRGERRRGRGHDGLYQGLSTATRDTYDALHMQ
ALPPR.

The abovementioned pans forming the chimeric antigen receptor of the present invention may be directly connected to each other, or may be connected through a linker sequence. The linker sequence may be a linker sequence suitable for antibodies well known in the art, for example, a linker sequence containing G and S Generally, the linker contains one or more motifs that repeat in front and back. For example, the motif can be GGGS, GGGGS, SSSSG, GGSSA, and GGSGG. Preferably, the motifs are adjacent in the linker sequence, and no amino add residues are inserted between the repeats. The linker sequence can include 1, 2, 3, 4 or 5 repeating motifs. A length of the linker can be 3-25 amino add residues, for example, 3-15, 5-15, 10-20 amino add residues. In certain embodiments, the linker sequence is a poly glycine linker sequence. The number of glycine in the linker sequence is not particularly limited, and is usually 2-20, such as 2-15, 2-10, and 2-8. In addition to glycine and serine, the linker can also contain other known amino add residues, such as alanine (A), leucine (L), threonine (T), glutamic acid (E), phenylalanine (F), arginine (R) and glutamine (Q).

It should be understood that it is often necessary to design suitable restriction sites in gene cloning operations, which inevitably introduce one or more irrelevant residues at a end of the expressed amino acid sequence without affecting the activity of the target sequence. In order to construct a fusion protein, promote expression of a recombinant protein, obtain a recombinant protein that is automatically secreted out of a host cell, or facilitate purification of the recombinant protein, it is often necessary to add certain amino adds to the N-terminal, C-terminal or other suitable regions of the recombinant protein, for example, including but not limited to suitable linker peptides, signal peptides, leader peptides, terminal extensions and die like. Therefore, the amino terminal or carboxy terminal of the fusion protein (that is, the CAR) of the present invention may also contain one or more polypeptide fragments as protein tags. Any suitable tag can be used in the present invention. For example, the tags can be FLAG, HA, HAl, c-Myc, Poly-His, Poly-Arg, Strep-TagII, AU1, EE, T7, 4A6, ε, B, gE and Tyl. These tags can be used topurifv proteins.

The polynucleotide sequence provided in the present invention is selected from:

(1) a polynucleotide sequence encoding the aforementioned chimeric antigen receptor; and

(2) a complementary sequence of the polynucleotide sequence as described in (1).

Preferably, the polynucleotide sequence is shown in SEQ ID NO: 15-18. Specifically:

(SEQ ID NO: 15, CD19 41BB KR CAR)
ATGGCCTTACCAGTGACCGCCTTGCTCCTGCCGCT
GGCCTTGCTGCTCCACGCCGCCAGGCCGGAGCAGA
AGCTGATCAGCGAGGAGGACCTGGACATCCAGATG
ACACAGACTACATCCTCCCTGTCTGCCTCTCTGGG
AGACAGAGTCACCATCAGTTGCAGGGCAAGTCAGG
ACATTAGTAAATATTTAAATTGGTATCAGCAGAAA
CCAGATGGAACTGTTAAACTCCTGATCTACCATAC
ATCAAGATTACACTCAGGAGTCCCATCAAGGTTCA
GTGGCAGTGGGTCTGGAACAGATTATTCTCTCACC
ATTAGCAACCTGGAGCAAGAAGATATTGCCACTTA
CTTTTGCCAACAGGGTAATACGCTTCCGTACACGT
TCGGAGGGGGGACCAAGCTGGAGATCACAGGTGGC
GGTGGCTCGGGCGGTGGTGGGTCGGGTGGCGGCGG
ATCTGAGGTGAAACTGCAGGAGTCAGGACCTGGCC
TGGTGGCGCCCTCACAGAGCCTGTCCGTCACATGC
ACTGTCTCAGGGGTCTCATTACCCGACTATGGTGT
AAGCTGGATTCGCCAGCCTCCACGAAAGGGTCTGG
AGTGGCTGGGAGTAATATGGGGTAGTGAAACCACA
TACTATAATTCAGCTCTCAAATCCAGACTGACCAT
CATCAAGGACAACTCCAAGAGCCAAGTTTTCTTAA
AAATGAACAGTCTGCAAACTGATGACACAGCCATT
TACTACTGTGCCAAACATTATTACTACGGTGGTAG
CTATGCTATGGACTACTGGGGCCAAGGAACCTCAG
TCACCGTCTCCTCAACCACCACTCCCGCACCCCGC
CCTCCTACTCCTGCCCCTACCATTGCTAGCCAACC
GCTTAGTCTGAGACCTGAGGCCTGTAGGCCCGCTG
CTGGTGGCGCTGTGCACACCCGAGGATTGGACTTC
GCTTGCGACATCTACATCTGGGCACCTCTGGCTGG
GACCTGCGGCGTGTTGTTGTTGAGCCTGGTGATTA
CGCTGTACTGTAGACGGGGCAGACGCAGACTCCTG
TATATATTCCGCCAACCATTTATGAGACCAGTACA
AACTACTCAAGAGGAAGATGGCTGTAGCTGCCGAT
TTCCAGAAGAAGAAGAAGGAGGATGTGAACTGAGA
GTGAGATTCAGCAGGAGCGCAGACGCCCCCGCGTA
CCAGCAGGGCCAGAACCAGCTCTATAACGAGCTCA
ATCTAGGACGAAGAGAGGAGTACGATGTTTTGGA
CCGCAGACGTGGCCGGGACCCTGAGATGGGGGGAA
GACCGcagAGAAGGCGCAACCCTCAGGAAGGCCTG
TACAATGAACTGCAGCGCGATAGAATGGCGGAGGC
CTACAGTGAGATTGGGATGAGAGGCGAGCGCCGGA
GGGGCAGAGGGCACGATGGCCTTTACCAGGGTCTC
AGTACAGCCACCCGCGACACCTACGACGCCCTTCA
CATGCAGGCCCTGCCTCCTCGC.
(SEQ ID NO: 16, CD19 CD28 KR CAR)
ATGGCCTTACCAGTGACCGCCTTGCTCCTGCCGCT
GGCCTTGCTGCTCCACGCCGCCAGGCCGGAGCAGA
AGCTGATCAGCGAGGAGGACCTGGACATCCAGATG
ACACAGACTACATCCTCCCTGTCTGCCTCTCTGGG
AGACAGAGTCACCATCAGTTGCAGGGCAAGTCAGG
ACATTAGTAAATATTTAAATTGGTATCAGCAGAAA
CCAGATGGAACTGTTAAACTCCTGATCTACCATAC
ATCAAGATTACACTCAGGAGTCCCATCAAGGTTCA
GTGGCAGTGGGTCTGGAACAGATTATTCTCTCACC
ATTAGCAACCTGGAGCAAGAAGATATTGCCACTTA
CTTTTGCCAACAGGGTAATACGCTTCCGTACACGT
TCGGAGGGGGGACCAAGCTGGAGATCACAGGTGGC
GGTGGCTCGGGCGGTGGTGGGTCGGGTGGCGGCGG
ATCTGAGGTGAAACTGCAGGAGTCAGGACCTGGCC
TGGTGGCGCCCTCACAGAGCCTGTCCGTCACATGC
ACTGTCTCAGGGGTCTCATTACCCGACTATGGTGT
AAGCTGGATTCGCCAGCCTCCACGAAAGGGTCTGG
AGTGGCTGGGAGTAATATGGGGTAGTGAAACCACA
TACTATAATTCAGCTCTCAAATCCAGACTGACCAT
CATCAAGGACAACTCCAAGAGCCAAGTTTTCTTAA
AAATGAACAGTCTGCAAACTGATGACACAGCCATT
TACTACTGTGCCAAACATTATTACTACGGTGGTAG
CTATGCTATGGACTACTGGGGCCAAGGAACCTCAG
TCACCGTCTCCTCAACCACCACTCCCGCACCCCGC
CCTCCTACTCCTGCCCCTACCATTGCtAGCCAACC
GCTTAGTCTGAGACCTGAGGCCTGTAGGCCCGCTG
CTGGTGGCGCTGTGCACACCCGAGGATTGGACTTC
GCTTGCGACATCTACATCTGGGCACCTCTGGCTGG
GACCTGCGGCGTGTTGTTGTTGAGCCTGGTGATTA
CGCTGTACTGTAGGAGTAGAAGGAGCAGGCTCCTG
CACAGTGACTACATGAACATGACTCCCCGCCGCCC
CGGGCCCACCCGCAGACATTACCAGCCCTATGCCC
CACCACGCGACTTCGCAGCCTATCGCTCCAGAGTG
AGATTCAGCAGGAGCGCAGACGCCCCCGCGTACCA
GCAGGGCCAGAACCAGCTCTATAACGAGCTCAATC
TAGGACGAAGAGAGGAGTACGATGTTTTGGACCGC
AGACGTGGCCGGGACCCTGAGATGGGGGGAAGACC
GcagAGAAGGCGCAACCCTCAGGAAGGCCTGTACA
ATGAACTGCAGCGCGATAGAATGGCGGAGGCCTAC
AGTGAGATTGGGATGAGAGGCGAGCGCCGGAGGGG
CAGAGGGCACGATGGCCTTTACCAGGGTCTCAGTA
CAGCCACCCGCGACACCTACGACGCCCTTCACATG
CAGGCCCTGCCTCCTCGC.
(SEQ ID NO. 17, GD2 41BB KR CAR)
ATGGCCTTACCAGTGACCGCCTTGCTCCTGCCGCT
GGCCTTGCTGCTCCACGCCGCCAGGCCGGAGCAGA
AGCTGATCAGCGAGGAGGACCTGGATGTTGTCATG
ACTCAAACCCCTTTATCTTTGCCCGTATCCCTTGG
TGACCAGGCTTCAATTTCGTGTCGTAGTAGCCAAT
CTCTCGTGCATCGCAATGGCAACACATATGTACAC
TGGTACCTGCAGAAACCAGGACAATCCCCGAAGTT
ATTGATCCATAAAGTTTCAAATCGATTTTCGGGGG
TCCCTGATCGGTTCAGTGGTAGCGGCTCTGGAACG
GACTTTACTCTTAAGATATCCAGAGTAGAAGCCGA
GGATCTCGGGGTGTATTTCTGCTCACAGTCGACCC
ACGTTCCCCCACTAACATTTGGTGCAGGCACGAAA
CTGGAATTAAAGGGTGGCGGTGGCTCGGGCGGTGG
TGGGTCGGGTGGCGGCGGATCTGAAGTTCAATTAT
TGCAGTCTGGTCCTGAGCTXGAAAAACCCGGCGCT
TCCGTCATGATTTCATGTAAGGCCTCGGGAAGTAG
CTTTACTGGGTATAATATGAACTGGGTACGTCAAA
ATATCGGTAAATCTCTCGAATGGATAGGCGCAATT
GATCCATACTATGGAGGGAOCTCCTACAACCAGAA
GTTCAAAGGTCGCGCGACACTAACGGTGGACAAGT
CATCGAGTACTGCTTATATGCATCTGAAAAGCTTA
ACCTCTGAAGATTCCGCCGTTTACTATTGCGTCTC
AGGCATGGAGTACTGGGGACAAGGGACATCGGTAA
CGGTGAGTAGCACCACCACTCCCGCACCGCGCCCT
CCTACTCCTGCCCCTACCATTGCTAGCCAACCGCT
TAGTCTGAGACCTGAGGCCTGTAGGCCCGCTGCTG
GTGGCGCTGTGCACACCCGAGGATTGGACTTCGCT
TGCGACATCTACATCTGGGCACCTCTGGCTGGGAC
CTGCGGCGTGTTGTTGTTGAGCCTGGTGATTACGC
TGTACTGTAGACGGGGCAGACGCAGACTCCTGTAT
ATATTCCGCCAACCATTTATGAGACCAGTACAAAC
TACTCAAGAGGAAGATGGCTGTAGCTGCCGATTTC
CAGAAGAAGAAGAAGGAGGATGTGAACTGAGAGTG
AGATTCAGCAGGAGCGCAGACGCCCCCGCGTACCA
GCAGGGCCAGAACCAGCTCTATAACGAGCTCAATC
TAGGACGAAGAGAGGAGTACGATGTTTTGGACCGC
AGACGTGGCCGGGACCCTGAGATGGGGGGAAGACC
GcagAGAAGGCGCAACCCTCAGGAAGGCCTGTACA
ATGAACTGCAGCGCGATAGAATGGCGGAGGCCTAC
AGTGAGATTGGGATGAGAGGCGAGCGCCGGAGGGG
CAGAGGGCACGATGGCCTTTACCAGGGTCTCAGTA
CAGCCACCCGCGACACCTACGACGCCCTTCACATG
CAGGCCCTGCCTCCTCGC.
(SEQ ID NO: 18, GD2 CD28 KR CAR)
ATGGCCTTACCAGTGACCGCCTTGCTCCTGCCGCT
GGCCTTGCTGCTCCACGCCGCCAGGCCGGAGCAGA
AGCTGATCAGCGAGGAGGACCTGGATGTTGTCATG
ACTCAAACCCCTTTATCTTTGCCCGTATCCCTTGG
TGACCAGGCTTCAATTTCGTGTCGTAGTAGCCAAT
CTCTCGTGCATCGCAATGGCAACACATATCTACAC
TGGTACCTGCAGAAACCAGGACAATCCCCGAAGTT
ATTGATCCATAAAGTTTCAAATCGATTTTCGGGGG
TCCCTGATCGGTTCAGTGGTAGCGGCTCTGGAACG
GACTTTACTCTTAAGATATCCAGAGTAGAAGCCGA
GGATCTCGGGGTGTATTTCTGCTCACAGTCGACCC
ACGTTCCCCCACTAACATTTGGTGCAGGCACGAAA
CTGGAATTAAAGGGTGGCGGTGGCTCGGGCGGTGG
TGGGTCGGGTGGCGGCGGATCTGAAGTTCAATTAT
TGCAGTCTGGTCCTGAGCTTGAAAAACCCGGCGCT
TCCGTCATGATTTCATGTAAGGCCTCGGGAAGTAG
CTTTACTGGGTATAATATGAACTGGGTACGTCAAA
ATATCGGTAAATCTCTCgaaTGGATAGGCGCAATT
GATCCATACTATGGAGGGACCTCCTACAACCAGAA
GTTCAAAGGTCGCGCGACACTAACGGTGGACAAGT
CATCGAGTACTGCTTATATGCATCTGAAAAGCTTA
ACCTCTGAAGATTCCGCCGTTTACTATTGCGTCTC
AGGCATGGAGTACTGGGGAGAAGGGACATCGGTAA
CGGTGAGTAGCACCACCACTCCCGCACCCCGCCCT
CCTACTCCTGCCCCTACCATTGCtAGCCAACCGCT
TAGTCTGAGACCTGAGGCCTGTAGGCCCGCTGCTG
GTGGCGCTGTGCACACCCGAGGATTGGACTTCGCT
TGCGACATCTACATCTGGGCACCTCTGGCTGGGAC
CTGCGGCGTGTTGTTGTTGAGCCTGGTGATTACGC
TGTACTGTAGGAGTAGAAGGAGCAGGCTCCTGCAC
AGTGACTACATGAACATGACTCCCCGCCGCCCCGG
GCCCACCCGCAGACATTACCAGCCCTATGCCCCAC
CACGCGACTTCGCAGCCTATCGCTCCAGAGTGAGA
TTCAGCAGGAGCGCAGACGCCCCCGCGTACCAGCA
GGGCCAGAACCAGCTCTATAACGAGCTCAATCTAG
GACGAAGAGAGGAGTACGATGTTTTGGACCGCAGA
CGTGGCCGGGACCCTGAGATGGGGGGAAGACCGca
gAGAAGGCGCAACCCTCAGGAAGGCCTGTACAATG
AACTGCAGCGCGATAGAATGGCGGAGGCCTACAGT
GAGATTGGGATGAGAGGCGAGCGCCGGAGGGGCAG
AGGGCACGATGGCCTTTACCAGGGTCTCAGTACAG
CCACCCGCGACACCTACGACGCCCTTCACATGCAG
GCCCTGCCTCCTCGC.

The polynucleotide sequence of the present invention may be in the form of DNA or RNA. The form of DNA includes cDNA, genomic DNA or synthetic DNA. DNA can be single-stranded or double-stranded. DNA can be a coding strand or a non-coding strand. The present invention may also include degenerate variants of the polynucleotide sequence encoding the fusion protein, that is, the nucleotide sequences that encode the same amino acid sequence but have different nucleotide sequences.

The polynucleotide sequences described herein can usually be obtained by PCR amplification. Specifically, a relevant sequence can be obtained by designing primers according to the nucleotide sequences disclosed herein, especially the open reading frame sequences, using a commercially available cDNA library or a cDNA library prepared by a conventional method known to those skilled in the art as a template and amplifying. When the sequence is relatively long, it is often necessary to perform two or more PCR amplifications, and then splice the amplified fragments together in a correct order.

A nucleic acid construct provided in the present invention contains the aforementioned polynucleotide sequence.

The nucleic add construct also includes one or more regulatory sequences operatively linked to the aforementioned polynucleotide sequence A coding sequence of the CAR of the present invention can be operated in a variety of ways to ensure the expression of proteins. The nucleic add construct can be operated according to different expression vectors or requirements before inserting into the vector. Techniques for altering polynucleotide sequences using recombinant DNA methods are known in the art.

The regulatory sequence can be a suitable promoter sequence. The promoter sequence is usually operatively linked to a coding sequence of a protein to be expressed. The promoter can be any nucleotide sequence that shows transcriptional activity in a selected host cell, including mutant, truncated and hybrid promoters, and can be obtained from genes encoding extracellular or intracellular polypeptides that homologous or heterologous with the host cell.

The regulatory sequence may also be a suitable transcription terminator sequence, which is a sequence recognized by the host cell to terminate transcription. The terminator sequence is operatively linked to a 3′-terminal of a nucleotide sequence encoding the polypeptide. Any terminator that functions in the selected host cell can be used in the present invention.

The regulatory sequence can also be a suitable leader sequence, which is an untranslated region of mRNA that is important for host cells' translation. The leader sequence is operatively linked to a 5′-terminal of the nucleotide sequence encoding the polypeptide. Any terminator that functions in the selected host cell can be used in the present invention.

Preferably, the nucleic acid construct is a vector.

The expression of the polynucleotide sequence encoding the CAR is usually achieved by operatively linking the polynucleotide sequence encoding the CAR to the promoter, and incorporating the construct into an expression vector. The vector may be suitable for replication and integration to eukaryotic cells. A typical cloning vector contains transcription and translation terminators, initiation sequences, and promoters that can be used to regulate the expression of the desired nucleic add sequence.

The polynucleotide sequence encoding the CAR of the present invention can be cloned into many types of vectors. For example, it can be cloned into plasmids, phagemids, phage derivatives, animal viruses and cosmids. Further, the vector is an expression vector. The expression vector can be provided to the cell in the form of 3 viral vector. Viral vector technology is well known in the art. Viruses that can be used as vectors include but are not limited to retrovirus, adenovirus, adeno-associated virus, herpes virus, and lentivirus. Generally, a suitable vector contains origins of replication, promoter sequences, convenient restriction enzyme sites, and one or more selectable markets that function in at least one organism.

More preferably, the nucleic acid construct is a lentiviral vector containing a replication initiation site, 3′LTR, 5′LTR and the aforementioned polynucleotide sequence

An example of suitable promoters is the immediate early cytomegalovirus (CMV) promoter sequence. The promoter sequence is a strong constitutive promoter sequence capable of driving high-level expression of any polynucleotide sequence operatively linked thereto. Another example of suitable promoters is elongation factor-1α (EF-1α). However, other constitutive promoter sequences can also be used, including but not limited to simian virus 40 (SV40) early promoters, mouse mammary tumor viruses (MMTV), human immunodeficiency virus (HIV) long terminal repeat (LTR) promoters, MoMuLV promoters, avian leukemia virus promoters, Epstein-Barr virus immediate early promoters. Rous sarcoma virus promoters, and human gene promoters, the human gene promoters such as but are not limited to actin promoters, myosin promoters, heme promoters and creatine kinase promoters. Further, the use of inducible promoters can also be considered. The use of inducible promoters provides a molecular switch that can turn on the expression of the polynucleotide sequence operatively linked to the inducible promoter when the expression is desirable, and turn off the expression when the expression is undesirable. Examples of inducible promoters include but are not limited to metallothionein promoters, glucocorticoid promoters, progesterone promoters and tetracydine promoters.

In order to evaluate the expression of the CAR polypeptide or part thereof, the expression vector introduced into the cell may also contain either or both of a selectable marker gene or reporter gene, so as to identify and select expressing cells from a cell population transfected or infected by viral vectors. In other aspects, the selectable marker can be carried on a single DNA segment and used in a co-transfection procedure. Both the selectable marker and reporter gene can be flanked by appropriate regulatory sequences, so as to be expressed in the host cell. Useful selectable markers include, for example, antibiotic resistance genes such as neo and the like.

Reporter genes are used to identify potentially transfected cells and to evaluate the functionality of regulatory sequences. After introducing DNA into a recipient cell, the expression of the reporter gene is measured at an appropriate time. Suitable reporter genes may include genes encoding luciferase, β-galactosidase, chloramphenicol acetyltransferase, secreted alkaline phosphatase, or green fluorescent protein genes. Suitable expression systems are well known and can be prepared using known techniques or obtained commercially.

Methods for introducing genes into cells and expressing genes into cells are known in the art. The vectors can be easily introduced into host cells such as mammalian, bacterial, yeast, or insect cells through any method in the art. For example, the expression vector can be transferred into the host cell by physical, chemical or biological methods.

Physical methods for introducing polynucleotides into host cells include calcium phosphate precipitation, lipofection, particle bombardment, microinjection, electroporation, and the like. Biological methods for introducing interested polynucleotides into host cells include the use of DNA and RNA vectors. Chemical methods for introducing polynucleotides into host cells include colloidal dispersion systems, such as macromolecular complexes, nanocapsules, microspheres, beads, and lipid-based systems, including oil-in-water emulsions, micelles, mixed micelles, and liposomes.

Biological methods for introducing polynucleotides into host cells include the use of viral vectors, especially lentiviral vectors, which has become the most widely used method for inserting genes into mammalian cells such as human cells. Other viral vectors can be derived from lentivirus, poxvirus, herpes simplex virus I, adenovirus, adeno-associated virus, and the like. Many virus-based systems have been developed for transferring genes into mammalian cells. For example, lentivirus provides a convenient platform for gene delivery systems. The selected gene can be inserted into the vector and packaged into lentiviral particles using techniques known in the art. The recombinant virus can then be isolated and delivered to target cells in vivo or in vitro. Many retroviral systems are known in the art. In some embodiments, adenoviral vectors are used. Many adenovirus vectors are known in the art In one embodiment, lentivirus vector are used.

The lentivirus provided in the present invention contains the aforementioned nucleic acid constructs. Preferably, the lentivirus contains the vectors. More preferably, the lentivirus contains the lentiviral vectors.

The present invention provides a method for activating T cells in vitro, which includes the step of infecting the T cells with the aforementioned lentivirus.

The CAR-T cells of Ac present invention can undergo a stable in-vivo T cell expansion, and maintain a high level in the blood and bone marrow for a prolonged amount of time, and form specific memory T cells. Without wishing to be bound by any specific theory, the CAR-T cell of the present invention can differentiate into a central memory-like state in vivo after encountering and subsequently eliminating a target cell expressing an alternative antigen.

The present invention also includes a type of cell therapy in which T cells are genetically modified to express the CAR described herein, and CAR-T cells are injected into subjects in need thereof. The injected cells can kill the tumor cells of the subjects. Unlike antibody therapy, CAR-T cells can replicate in vivo, producing a long-term persistence that can lead to sustained tumor control.

The anti-tumor immune response caused by CAR-T cells can be an active or passive immune response. In addition, the CAR-mediated immune response can be a part of an adoptive immunotherapy, in which CAR-T cells induce an immune response specific to an antigen-binding domain of the CAR.

The treatable cancer may be a non-solid tumor, for example, a hematological tumor, such as leukemia and lymphoma. In particular, the diseases that can be treated with the CAR, coding sequences thereof, nucleic acid constructs, expression vectors, viruses and CAR-T cells of the present invention are preferably CD19-mediated diseases, especially CD19-mediated hematological tumors.

Specifically, as used herein. “CD19-mediated diseases” comprise but arc not limited to leukemia and lymphoma, such as B-cell lymphoma, mantle cell lymphoma, acute lymphocytic leukemia, chronic lymphocytic leukemia, hairy cell leukemia, and acute myeloid leukemia.

A genetically modified T cell or a pharmaceutical composition containing the genetically modified T cell provided in the present invention contains the aforementioned polynucleotide sequences, or contains the aforementioned nucleic acid constructs, or is infected with the aforementioned lentivirus, or is prepared by the aforementioned methods.

The CAR-modified T cells of the present invention can be administered alone or as a pharmaceutical composition in combination with diluents and or other components such as related cytokines or cell populations. Briefly, the pharmaceutical composition of the present invention may include the CAR-T cells as described herein in combination with one or more pharmaceutically or physiologically acceptable vectors, diluents or excipients. Such compositions may include buffers such as neutral buffered saline, sulfate buffered saline and the like; carbohydrates such as glucose, mannose, sucrose or dextran, mannitol, proteins, polypeptides or amino acids such as glycine; antioxidants, chelates such as EDTA or glutathione: adjuvants (for example, aluminum hydroxide); and preservatives.

The pharmaceutical composition of the present invention can be administered in a manner suitable for the disease to be treated (or prevented). The number and frequency of administration will be determined by factors such as the patient's condition, and the type and severity of the patient's disease

When referring to “immunologically effective amount”, “anti-tumor effective amount”, “tumor-suppressive effective amount” or “therapeutic amount”, the precise amount of the composition of the present invention to be administered can be determined by die physician, taking the age, weight, tumor size, degree of infection or metastasis, and individual differences in condition of a patient (subject) into consideration. It may generally be pointed out that the pharmaceutical composition including the T cells described herein may be administered at a dose of 10⁴ to 10⁹ cells/kg body weight, preferably 10⁵ to 10⁵ cells/kg body weight. The T cell composition can also be administered multiple times at these doses. The cells can be administered using injection techniques well-known in immunotherapy. The optimal dosage and treatment regimen for a specific patient can be easily determined by those skilled in the medical field through monitoring the patient's signs of disease and adjusting the treatment accordingly.

The administration of the object composition can be performed in any convenient manner, including by spraying, injection, swallowing, infusion, implantation, or transplantation. The compositions described herein can be administered to patients subcutaneously, intracutaneously. intratumorally, intranodallv, intraspinally, intramuscularly, by intravenous injection, or intraperitoneally. In one embodiment, the T cell composition of the present invention is administered to the patient by intradermal or subcutaneous injection. In another embodiment, the T cell composition of the present invention is preferably administered by intravenous injection. The T cell composition can be injected directly into tumors, lymph nodes, or sites of infection.

In some embodiments of the present invention, the CAR-T cell of the present invention or a composition thereof can be combined with other therapies known in the art. Such therapies include but are not limited to chemotherapies, radiotherapies, and immunosuppressive agents. For example, it can be used for therapy in combination with various radiotherapy preparations, including: cydosporine, azathioprine, methotrexate, mycophenolate, FK506, fludarabine, raparaycin, mycophenolic acid, and the like. In a further embodiment, the cell composition of the present invention is administered to patients in combination (for example, before, simultaneously, or after) with bone marrow transplantation: T cell ablation therapy using chemotherapeutic agents such as fludarabine, external beam radiotherapy (XRT), cydophosphamide, or antibodies such as OKT3 or CAMPATH.

As used herein, “anti-tumor effect” refers to a biological effect, which can be represented by a decrease in tumor volume, a decrease in the number of tumor cells, a decrease in the number of metastases, an increase in life expectancy, or the improvement of various physiological symptoms related to cancer.

“Patient”, “subject”, “individual” and tire like may be used interchangeably herein and refer to a living organism that can be caused an immune response, such as mammals. Examples include but are not limited to humans, dogs, cats, mice, rats and transgenic species thereof.

Use of the aforementioned chimeric antigen receptor, the aforementioned polynucleotide sequence, the aforementioned nucleic acid construct or the aforementioned lentivirus in preparing activated T cells and or inhibiting T cell degradation.

Use of the aforementioned chimeric antigen receptor, the aforementioned polynucleotide sequence, the aforementioned nucleic acid construct, the aforementioned lentivirus or the aforementioned genetically modified T cell in any one or more of die following application (1) preparing tumor therapeutic drugs; (2) improving tumor killing efficiency; (3) maintaining T cell proliferation capacity; and (4) inhibiting tumor development

Preferably, the tumor is selected from one or more of leukemia or lymphoma

More preferably, the tumor is selected from B-cell lymphoma, mantle cell lymphoma, acute lymphocytic leukemia, chronic lymphocytic leukemia, hairy cell leukemia, and acute myeloid leukemia.

Embodiments of the present invention will be described below with specific examples. Those skilled in the art can easily understand other advantages and effects of the present invention from the content disclosed in this specification. The present invention can also be implemented or applied through other different specific embodiments, and various details in this specification may also be modified or changed based on different viewpoints and applications without departing from the spirit of the present invention.

Before further describing specific embodiments of the present invention, it should be understood that, the protection scope of the present invention is not limited to the following specific embodiments; and it should be further understood that, terms used in the examples of the present invention are used for describing the specific embodiments, and ate not intended to limit the protection scope of the present invention: and in the specification and claims of the present invention, the singular forms “one”, “a/an” and “the” are intended to include the plural forms, unless the context clearly indicates otherwise.

When value ranges are described in the examples, it should be understood that, the two endpoints of each value range and any value between two endpoints may be selected unless the present invention describes otherwise. Unless otherwise defined, all technical and scientific terms used herein have the same meaning as commonly understood by those skilled in the technical field. In addition to the specific methods, devices and materials used in the examples, any method, device and material in the related art similar with or equivalent to the methods, devices and materials in the examples may further be used to realize the present invention according to comprehension of those skilled in the technical field for the related art and recordings of the present invention.

Unless otherwise specified, the experimental methods, detection methods, and preparation methods disclosed in the present invention all adopt conventional technologies such as molecular biological, biochemical, chromatin structure and analysis, analytical chemical, cell culture, and recombinant DNA technologies in the technical field and conventional technologies in related fidds

### EXAMPLE 1

### Vector Construction of CAR

The sequences of antigen-specific single chain antibody (scFv) of CD19 and GD2 CAR as used in the present invention are derived from clinically used FMC63 and 14g2a sequences, respectively. An extracellular segment structure of CAR is composed of CD8α signal peptide sequence, myc tag sequence, scFv sequence, and CD8α hinge sequence in series; a transmembrane region sequence is a transmembrane region sequence of CD8α; an intracellular segment structure is composed of human 41BB intracellular segment sequence or CD28 intracellular segment sequence connected in series with human CD3ζ intracellular segment sequence. The above-mentioned amino add sequences and the amino add sequences where lysine in the intracellular segment was mutated to arginine were all converted into base sequences via codon optimization and synthesized by Qinglan Biotech. Except that the CAR used for the fluorescent confocal microscope experiment was doned into a pHR-hEF1α-EGFP vector, the base sequence of all CARs in the present invention was finally cloned into a pHR-hEF1α-IRES-EGFP vector by Gibson Assembly (NEB *E2611L).

### EXAMPLE 2

### Culture and Lentivirus Infection of Human Primary T Cell

All of the human primary T cells were taken from healthy and informed volunteers. Primary T cells were cultured in RPMI-1640 medium containing 10% fetal bovine serum, 100 U/ml penicillin and 100 μg/ml streptomycin sulfate (all the above reagents were purchased from Gibco). In order to maintain the proliferation of T cells, 100 U/ml of hIL-2 (Sigma-Aldrich) was added to the medium.

Preparation of lentivirus: 2.2×10⁵ Lenti-X 293T cells (TaKaRa #632180) were resuspended in DMEM medium (Gibco #11995-065) containing 10% fetal bovine serum and cultured in a 6-well cell culture plate (Corning #CLS3516) for 24 h. 500 ng of lentiviral packaged plasmids pCMVdR8.92 (Addgene #8455) and pMD2.G (Addgene #12259), and 500 ng of lentiviral plasmids to be packaged were added to the Lenti-X 293T cell culture medium using a liposome transfection system (Mirus #2300) according to the procedures of the protocols for liposome transfection After 48 h. the cell supernatant was collected and stored in a refrigerator at −80° C. for later use.

Lentiviral infection of primary T cells: magnetic beads coated with anti-CD3 and anti-CD28 antibodies (Life Technologies #11132D) were used to activate T cells. The T cells and the magnetic beads were co-incubated at a ratio of 1:3 for 24 h and then the prepared lentivirus was added for infection After 18 h. the cell supernatant was removed and replaced with fresh T cell complete medium. After the T cells were challenged by the magnetic beads for 5 days, the magnetic beads was removed and fresh T cell complete medium was supplemented every 2-3 days.

### EXAMPLE 3

### Flow Cytometry Analysis

Staining for cell surface markers an antibody was diluted in FACS buffer (phosphate buffer PBS+2% fetal bovine serum; and then co-incubated with cells for 25 min in the dark at 4° C.

Staining for intracellular markers: the cells were first fixed with 4% paraformaldehyde (Meilunbio #MA0192) at room temperature for 15 minutes, and then membranes were ruptured on ice for 50 min using pre-chilled methanol. After that, the cells were co-incubated with an antibody dilution (same with that in staining for surface markers) for 60 min in the dark at room temperature for staining. Zombie Violet Fixable viability kit (Biolegend #423114) was used to distinguish dead and alive states of the cells.

Data of flow cytometry was obtained by BD LSRFortessa machine (BD bioscience), and Flow Jo software (Tree Star) was used for data analysis. An information list of antibodies used in flow cytometry is as follows.

| Antibody | Clone | | |
|---|---|---|---|
| recognition site | number | Fluorescence | Supplier |
| Myc-tag | 9B11 | Alexa Flour 647 | CST |
| CD3 | HIT3a | APC | biolegend |
| CD3 | UCHT1 | PE/Cy7 | eBioscience |
| CD4 | RPA-T4 | BV605 | biolegend |
| CD8a | RPA-T8 | PerCP/Cy5.5, | biolegend |
| | | APC/Cy7 | |
| CD27 | O323 | Alexa Fluor 647 | biolegend |
| CD45RA | HI100 | PE/Cy7 | biolegend |
| CD45RO | UCHL1 | PE/Cy7 | biolegend |
| CD62L | DREG-56 | PE | biolegend |
| CD69 | FN50 | PE | biolegend |
| CD223 (LAG-3) | 3DS223H | PE/Cy7 | eBioscience |
| CD279 (PD-1) | NAT105 | APC | biolegend |
| CD279 (PD-1) | eBioJ105 (J105) | PE | eBioscience |
| CD366 (Tim-3) | F38-2E2 | PerCP/Cy5.5 | biolegend |
| IL-2 | MQ1-17H12 | APC, PE | biolegend |
| IFN-γ | 4S.B3 | Alexa Fluor 647 | biolegend |
| Granzyme B | GB11 | Alexa Fluor 647 | biolegend |
| CD19 | HIB19 | FITC, PE | biolegend |
| Ganglioside GD2 | 14G2a | PE | biolegend |

### EXAMPLE 4

### Fluorescence Confocal Imaging

Lysosome and mitochondrial imaging of cells was carried out in a state of living cells. LvsoTracker Red (Invitrogen #L7528) and MitoTracker Orange (Invitrogen #M7510) were used for staining and labeling of lysosome and mitochondria, respectively. A staining method was as follows dye was added to a culture medium at a dilution ratio of 1:1000 and co-incubated with the cells for 30 min in a 37° C. incubator. Cell imaging was obtained by AlR-si microscope (Nikon) or TCS SP8 STED microscope (Leica).

Staining for tumor tissue slice: the sliced tumor tissue was first immersed in 4% paraformaldehyde, and then fixed overnight at 4° C. The fixed tumor tissue was placed in a 30% sucrose solution for dehydration until the tissue mass was sunk to the bottom, and then the tissue was fixed and embedded with an OCT embedding agent (SAKURA #4583). The embedded tissue was sliced into 8 μm tissue slices with a freezing microtome (Leica) and then stained. The nucleus was identified by staining with Hoechst (Invitrogen #H1399). A staining method was as follows: dye was added to the PBS buffer at a dilution ratio of 1:1000, dropped on the slices and incubated for 10 min in the dark at room temperature, and the excess dye was washed away with the PBS buffer. CAR-T cells had EGFP fluorescence, and target cells had mCherry fluorescence, so that the two types of cells can be directly identified under a microscope. 70% glycerol was dropped on the slices, slides were covered and edges of the slides were sealed with nail polish to complete slice preparation. Tissue slice imaging was obtained by a TCS SP8 STED microscope.

### EXAMPLE 5

### Cell Metabolism Analysis

The mitochondrial function of CAR-T cells was tested by using an Seahorse Bioscience XF24 Extracellular Flux Analyzer. Firstly, Poly-L-lysine (Sigma-Aldrich #P4832) was coated in an XF24 cell culture plate, and then the CAR-T cells were resuspended in RPMI 1640 medium containing 2 mM L-glutamine, 5.5 mM glucose and 1 mM sodium pyruvate at a density of 1.5*10⁵ cells per well and cultured for 30 min. After that, the XF24 Extracellular Flux Analyzer was calibrated, and a calibration procedure referred to the manual of this instrument. At the same time, the CAR-T cells were placed in an incubator without CO₂ for incubation. The oxygen consumption rate (OCR) of the cells was determined by using 1 μM oligomydn, 2 μM 2.4-dinitrophenol, 40 nM rotenone and 1 μM antimycin A (the above inhibitors were all from Seahorse Bioscience).

### EXAMPLE 6

### Down-Regulation and Degradation Detection of CAR

CAR-T cells were co-incubated with target cells or non-target cells in a 24-well cell culture plate at a cell ratio of 1:3, and the incubation time was determined according to specific experiments. The down-regulation of CAR on a cell surface was determined via analyzing the expression of CAR on the cell surface by flow cytometry, the degradation of CAR was determined via analyzing the changes in the expression of intracellular and extracellular CAR by flow cytometry after rupture operation and further fixation. The detection of a CAR degradation pathway was performed by co-incubating the CAR-T cells and the target cells in a 96-well cell culture U bottom plate at a cell ratio of 1:3 for 8 h, in which MG132 (Selleck #S2619) and NH₄Cl (Sigma #254134) were used to inhibit the function of proteasome and lysosome, respectively. The detection of CAR degradation through westernblot was carried out by co-incubating the CAR-T cells and the target cells in a 48-well cell culture plate at a cell ratio of 1:1. In order to reduce the interference of newly generated CARs on the detection results, 25 μg/ml Cycloheximide (CST #2112) was also added to inhibit protein synthesis in some experiment.

### EXAMPLE 7

### Detection of In-Vitro Killing Function of CAR-T Based on Flow Cytometry

Double positive K562 target cells that express CD19 and mCherry fluorescence and K.562 non-target cells that do not express CD19 and mCherry fluorescence were mixed at a cell ratio of 1:1 and then co-incubated with CAR-T cells at a cell ratio according to the specific experimental arrangement for 3 days. The cells were cultured in a 24-well cell culture plate and 50 U ml IL-2 was added. Flow cytometry analysis: the mixed K562 cell population without T cells was used as a control group, and the proportion (CK%) of the K562 target cells in the mixed K562 cell population was analyzed and obtained: in an experimental group containing T cells, the T cells were distinguished from the K562 cell population by CD3Å staining, and then the proportion (EX%) of the K562 target cells in the K562 cell population was analyzed. The killing efficiency of the T cells =(1−EX%/CK%)×100%.

### EXAMPLE 8

### Mouse Tumor Model and In Vivo Function Test of CAR-T

The subjects for in-vivo experiments were 5 to 8-week-old immunodeficient (NSG) mice. In order to compare the in-vivo anti-tumor effects of CAR-T, K562-CD19 target cells expressing the firefly lucifcrase gene was firstly subcutancously inoculated into the outer thigh of NSG mice at a specified number based on specific experiments. After the target cells were stable in the body for 4 days, the tail vein of NSG mice was injected with CAR-T cells at a specified number based on specific experiments for treatment. After that, the development of tumors in the body was monitored by an in-vivo imaging technology every week. Specific procedure: a firefly luciferin substrate was intraperitoneally injected into tumor-bearing mice at a dose of 1.5 mg/g mouse body weight; after 10 minutes, when the substrate was fully circulated to the whole body of the mice, the mice were anesthetized with 2.5%-3.5% isoflurane gas for imaging. Bioluminescence imaging was performed by an IVIS Spectrum imaging system (Perkin Elmer), and fluorescence quantitative data was obtained by an in-vivo imaging software (Perkin Elmer).

### EXAMPLE 9

### Result Analysis

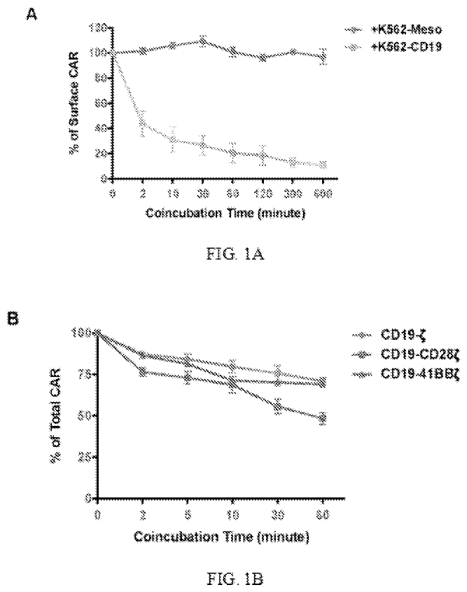

The results of the down-regulation and degradation of CAR-T under antigen challenge were shown in FIG. 1. Specifically, as shown in FIG. 1A, the ratio of the T cells and the K562 cells was 1:3, and the reaction time was shown on the abscissa. The expression amounts of CAR on the cell surface were analyzed through FACS When CAR-T interacted with the target cell K562-CD19, CAR was rapidly down-regulated, while it was not down-regulated when interacted with the non-target cell K562-Meso. As shown in FIG. 1B, the ratio of the T cells and the target cells was 1:3, and the reaction time was shown on the abscissa. The changes of total CAR expression amounts in cells were analyzed using FACS through intracellular staining. CARs have been degraded within 60 minutes under target cell challenge, and CARs containing different intracellular segment structures had different degradation rates.

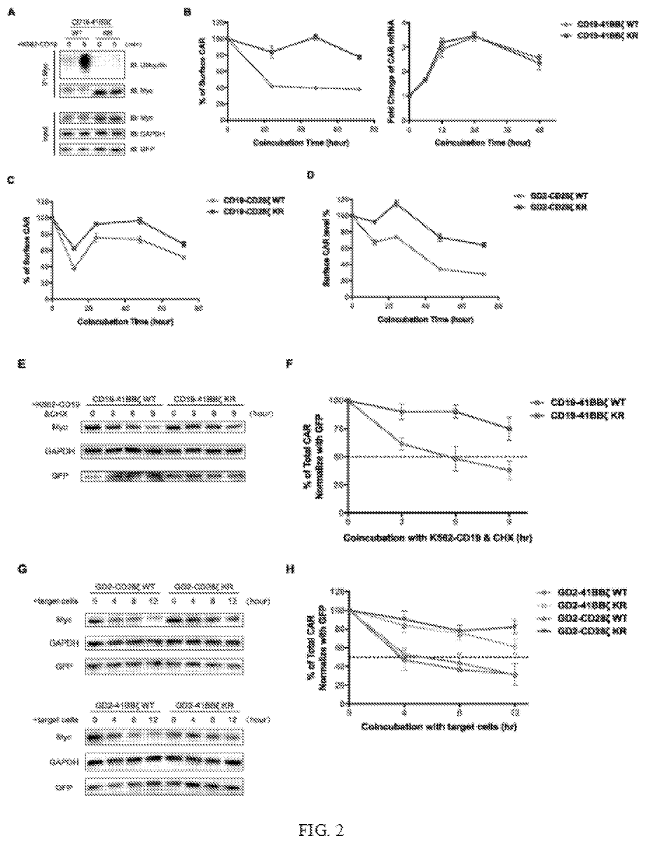

The results of down-regulation and degradation inhibited by KR-rautatcd CAR w ere shown in FIG. 2. Specifically, as shown in FIG. 2A, the CAR protein was pulled down through co-immunoprecipitation and detected by westernblot. An obvious ubiquitin-modified band was formed 5 minutes after WT CAR interacted with the target cells, while KR CAR could not be modified by ubiquitin before and after challenge, showing that mutation of the lysine site in the intracellular segment of CAR could effectively block the ubiquitin modification of CAR. As shown in FIG. 2B, the ratio of the T cells and the target cells was 1:3, and the reaction time was shown on the abscissa. The expression amounts of CAR on the cell surface were analyzed through FACS. After WT CAR-T interacted with the target cells, the CAR had a significant down-regulation response, while KR-mutated CAR was not sensitive to down-regulation; the mRNA changes of WT CAR and KR CAR were detected by fluorescence quantitative PCR, which showed that there was no difference in the amount of newly generated CAR between them, that is. the lack of KR CAR down-regulation was not due to the generation of more CARs but due to the inhibition of CAR down-regulation. As shown in FIG. 2C and FIG. 2D, the ratio of the T cells and the target cells was 1:3, and the reaction time was shown on the abscissa. The changes of the CAR expression amounts on the cell surface were analyzed through FACS. Although the down-regulation kinetics of the two CARs were different, the KR mutation could inhibit the down-regulation level of CAR According to the experiments of FIG. 2B-2D, the present invention has verified that the mutation of the lysine site in the intracellular segment of the CAR into arginine could effectively inhibit the down-regulation of the CAR mediated by antigen challenge, and could be suitable for CARs with different structures. As shown in FIG. 2E, the ratio of the T cells and the target cells was 1:1, and the reaction time was 3 to 9 h. In order to avoid the influence of newly generated CAR on the test results, 25 μM Cycloheximide (CHX) was added to the reaction system to inhibit protein synthesis. The changes of CAR protein expression amounts were detected through westernblot. The quantitative results of FIG. 2E were shown in FIG. 2F, which in (Seated that the KR mutation effectively inhibited the degradation of CAR in this reaction. As shown in FIG. 2G, the ratio of the T cells and the target cells was 1:1, and the reaction time was 4 to 12 h. The changes of CAR protein expression amounts were detected through westernblot. The quantitative results of FIG. 2G were shown in FIG. 2H. which indicated that the KR mutation effectively inhibited the degradation of CAR mediated by antigen challenge. According to the experiments of FIG. 2E-2H, the present invention has verified that the mutation of the lysine site in the intracellular segment of the CAR into arginine could effectively inhibit the degradation of the CAR mediated by antigen challenge, and could be suitable for CARs with various structures.

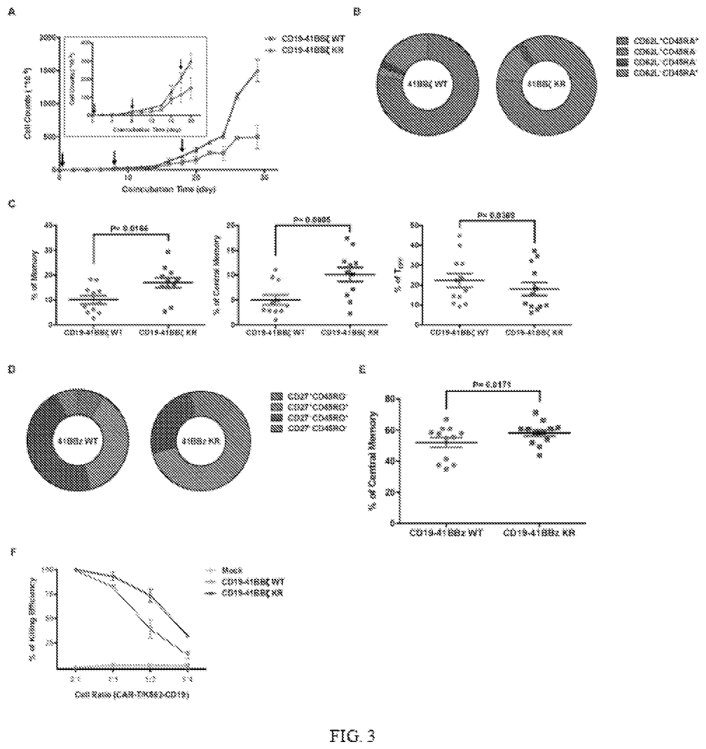

The effect of KR mutation on the in-vitro function of CAR-T was shown in FIG. 3. Specifically, all of the in-vitro functional experiments were performed on human primary T cells. As shown in FIG. 3A, CAR-Ts were co-incubated with irradiated target cells at a ratio of 2:1. T cells were counted every 2 days to maintain the cell density at 1.5 million/ml by supplementing new medium, and the target cells were supplemented at a cell ratio of 2:1 every 8-10 days. Taking CD19-41BBCCAR as an example, KR-mutated CAR-T could more effectively respond to the proliferation mediated by target cell challenge. The differentiation of WT and KR CAR-T under in-vitro culture conditions was shown in FIG. 3B, and FACS analysis was performed with CD62L and CD45RA as differentiation indicators. FIG. 3C was a statistical analysis of FIG. 3B, and KR mutation allowed CAR-T to differentiate more into central memory T cells and reduced the differentiation into terminal effector T cells. The differentiation phenotype results of CAR-T after being challenged by target cells for 4 days were shown in FIG. 3D, in which CAR-T and irradiated target cells were co-incubated at a ratio of 2:1, and FACS analysis was performed with CD27 and CD45RO as differentiation indicators. The statistical analysis of FIG. 3D was shown in FIG. 3E, and KR mutation allowed CAR-T to form more central memory T cells in response to target cell challenge As shown in FIG. 3F, the T cells and the target cells were co-incubated with the ratio shown on the abscissa. The incubation time was 3 days. The remaining ratio of the target cells was analyzed through FACS to calculate the killing efficiency of CAR-T. When the ratio of the T cells and the target cells was less than 1:1, KR CAR-T was considered to have higher killing efficiency.

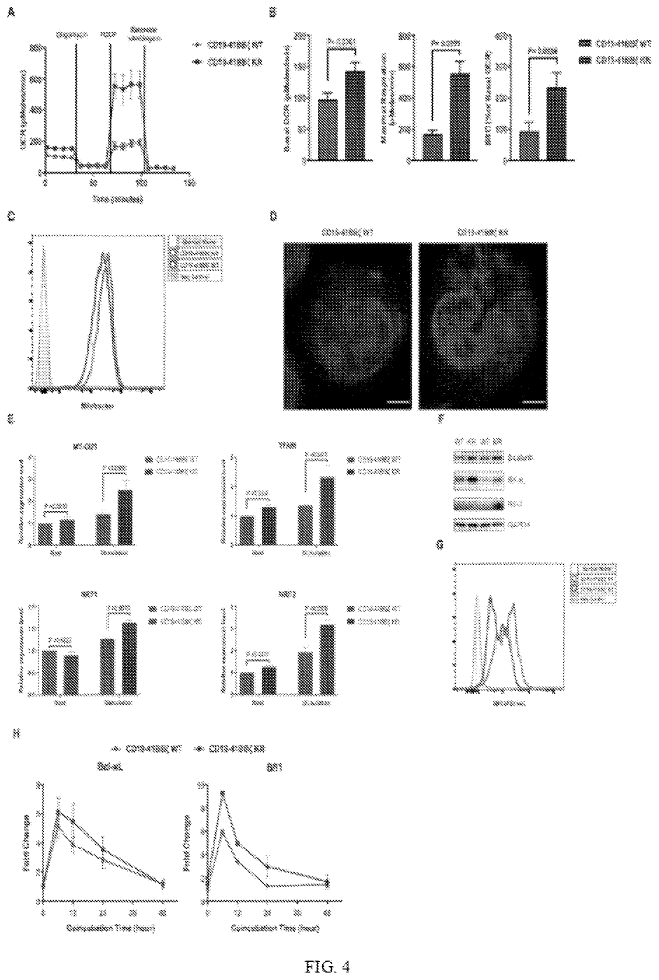

The molecular basis of CD19-41BBζKR CAR which can accumulate more central memory T cells and enhanced proliferation phenotypes, was shown in FIG. 4. This part of experiment was performed and verified on human primary T cells. As shown in FIG. 4A, the present invention used a Seahorse machine to detect the difference in oxidative metabolism between WT CAR-T and KR CAR-T, and the detected cells were first challenged by target cells for two weeks The statistical analysis of main parameters in FIG. 4A was shown in FIG. 4B. Compared with WT CAR-T. KR CAR-T had a higher basal oxygen consumption rate (OCR); KR CAR-T had a significantly higher maximum respiration capacity than that of WT CAR-T, finally, KR CAR-T also had a significantly higher remaining respiration capacity than that of WT CAR-T. This reflected that KR CAR-T had a stronger oxidative respiration metabolism, and KR CAR-T could perform oxidative phosphorylation more effectively even under hypoxic conditions. As shown in FIG. 4C and FIG. 4D, the present invention used mitotracker as a detection marker to verify that KR CAR-T had stronger mitochondrial production capacity using the FACS and fluorescence confocal detection methods, respectively. As shown in FIG. 4E, the present invention used fluorescence quantitative PCR to detect changes of four important genes related to mitochondrial production and function in WT and KR CAR-T before and after being challenged by irradiated target cells. The gene expression amount of unchallenged WT CAR-T was used as a control KR CAR-T after being challenged by the target cells showed higher expression levels than that of WT CAR-T in terms of all the four genes, which explained why KR CAR-T had a stronger mitochondrial production capacity The present invention explained the reason why KR CAR-T had stronger oxidative respiration capacity through the experiment of FIG. 4C-4E. KR CAR-T was verified to be capable of producing more anti-apoptotic factor Bcl-xL after being challenged by the target cells through westernblot and FACS detection methods, respectively. The results were shown in FIG. 4F and FIG. 4G. As shown in FIG. 4F, the westernblot of the present invention also reflected that KR CAR-T could produce more anti-apoptotic factor Bcl-2 and cell sternness marker β-catcnin. As shown in FIG. 4H, the present invention used fluorescence quantitative PCR as detective methods to verify that, compared with WT CAR-T, KR CAR-T produced more mRNA of 41BB downstream anti-apoptotic products Bd-xL and Bfl1 after being challenged by the target cells, which partially explained why KR CAR-T had a stronger proliferation capacity.

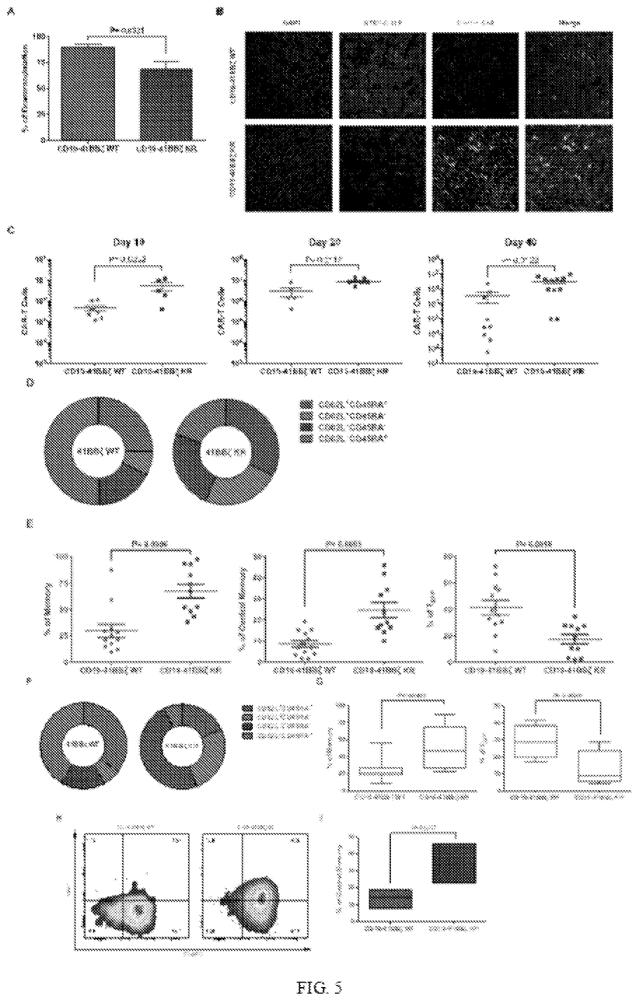

The comparison of the in-vivo functions of CD19-41BBCWT and KR CAR-T in tumor mouse models was shown in FIG. 5. Specifically, as shown in FIG. 5A, the present invention compared down-regulation performance of WT and KR CAR in vivo. The expression of CAR in peripheral blood and spleen of each mouse was used as a control to compare the changes of the expression level of CAR in the tumor the CARs were almost completely down-regulated on the WT CAR-T surface, while the KR mutation inhibited the down-regulation of CAR in the tumor, and more than 25% of CARs remained on the surface of CAR-T As shown in FIG. 5B, 14 days after injection of T cells, the tumor tissue was stripped from the mice and frozen slices were prepared for immunofluorescence confocal analysis. More CAR-T cells and fewer tumor cells appeared in the tumors of mice inoculated with KR CAR-T. As shown in FIG. 5C. the present invention prodded the changes of WT and KR CAR-T number in mouse spleen 10 days, 20 days, and 40 days after injection of CAR-T cells into tumor-bearing mice. KR CAR-T showed a more cell number than WT CAR-T at each detection time point, and the cell number did not decrease significantly from 10-40 days, while the cell number of WT CAR-T was significant reduced at day 40. The differentiation phenotype comparison of CAR-T in the spleen of tumor-bearing mice was shown in FIG. 5D. FACS analysis was performed with CD62L and CD45RA as differentiation indicators. The statistical analysis of FIG. 5D was shown in FIG. 5E. Compared with WT CAR, KR CAR promoted T cells to differentiate more into memory cells, especially central memory T cells, and reduced the differentiation into terminal effector T cells. Similarly, the differentiation phenotype comparison of CAR-T in the peripheral blood of tumor-bearing mice was shown in FIG. 5F. The statistical analysis of FIG. 5F was shown in FIG. 5G, and the conclusion was stilt that KR CAR promoted T cells to differentiate more into memory cells. The comparison result of differentiation phenotype of CAR-T in tumor tissue was shown in FIG. 5H. FACS analysis was performed with CD27 and CD45RO as differentiation indicators. The statistical analysis of FIG. 5H was shown in FIG. 5I. Compared with WT CAR-T, KR CAR-T could accumulate more central memory T cells. FIG. 5D-5I of the present invention largely explained the reason why KRCAR-T could maintain the proliferation capacity and more effectively infiltrate nun or tissues in tumor-bearing mice.

The comparison of the anti-tumor effects of CD19-41BBζ WT and KR CAR-T was shown in FIG. 6. Specifically, the development of target cells expressing a firefly luciferase gene in NSG mice injected with different T cells was shown in FIG. 6. 4 days after subcutaneous inoculation of 1 million target cells, 500,000 CAR-T or T cells that not infected with CAR-T were injected via tail vein for treatment. A luciferin substrate was intraperitoneally injected every other week and a fluorescence imaging system was used to monitor tumor development. The quantitative results of FIG. 6A were shown in FIG. 6B: ordinary T cells could not control tumor development; WT CAR-T could delay tumor development and achieve partial cure; and KR CAR-T could completely inhibit tumor development.

The effect of KR mutation on phenotype of CD19-41BBζ and CD19-CD28ζ CAR-T cells was shown in FIG. 7. Specifically, as shown in FIG. 7A and FIG. 7B, CD19 CARs containing 41BB, CD28 and KR-mutated costimulatory domains thereof were expressed on Jurkat cells by means of lentivirus infectiona and the expression level of the activation marker on cell surface, i.e. CD69 and ICOS, was detected by means of FACS after one week. It could be seen from the figure that the self-activation level of the mutated 41BB did not increase greatly and was lower than the self-activation level of normal CD28, while the mutated CD28 produced excessively strong self-activation signals, which would cause activation induced cell death (AICD) and accelerate the differentiation and exhaustion of cells, which was not conducive to the anti-tumor function of CAR-T cells The analysis results of the T cell exhaustion marker were shown in FIG. 7C. CD19 CARs containing 41BB, CD28 and KR-mutated costimulatory domains thereof were expressed on human primary T cells by means of lentivirus infection, and the expression level of PD-1 marker on the cell surface was detected by means of FACS after one week. The relative expression amounts were shown in the figure using the expression amounts of CD19-41BBζ WT CAR-T cells as a reference. It can be seen from the figure that there was no significant change in the expression of PD-1 for the mutated 41BB, while the mutated CD28 caused a significant increase in PD-1, which was not conducive to the function of CAR-T cells. The analysis results of T cell differentiation phenotype were shown in FIG. 7D, FIG. 7E. FIG. 7F, and FIG. 7G, in which CD62L/CD45RA was used as a differentiation marker, CD62L+CD45RA+ was used as a naive T cell. CD62L+CD45RA− was used as a central memory T cell, CD62L−CD45RA− was used as an effector memory T cell, CD62L−CD45RA+ was used as an effector memory T cell, and die degree of differentiation was increased sequentially. The CD19 or GD2 CAR containing CD28 and KR-mutated costimulatory domains thereof were expressed on human primary T cells by means of lentivirus infection, and the expression level of CD62L/CD45RA on the cell surface was deteaed by means of FACS after 10 to 14 days. It can be seen from FIG. 7D, FIG. 7E, FIG. 7F, and FIG. 7G that the KR mutation of CD28 led to enhanced differentiation, which was not conducive to the proliferation of CAR-T cells, whether on CD19 or GD2 CAR-T cells.

The comparison of effects of CD19-41BBζ WT and KR CAR-T in a tumor cell rechallenge model was shown in FIG. 8. Specifically, as shown in FIG. 8A, 4 days after subcutaneously inoculating 2×10⁶ tumor cells in NSG mice, 1.5×10⁴ CAR-T cells were injected into the tumor-bearing mice by tail vein injection for intervention treatment, and then the tumor development in the mice was monitored by an in-vivo imaging system every week. After 4 weeks, the tumor cells in only 2 mice injected with WT CAR-T cells were completely eliminated (5 in total), while 4 mice injected with KR CAR-T cells showed no signs of tumors (5 in total). After that, 1×10⁶ tumor cells were re-injected into the recovered mice (the black arrow points to the time of re-inoculation). In order to avoid the interference of tumor regeneration in situ, systemic inoculation was performed by tail vein injection. In follow-up tests, only one of the four recovered mice injected with KR CAR-T cells could not resist the re-inoculated tumor cells, while the remaining two mice injected with WT CAR-T cells all died of the rechallenge of tumor cells. In order to ensure that the two kinds of CAR-T cells act on tumor-bearing models at the same initial level, the present invention also compared the response of the CAR-T challenged by target cells in vitro to tumor cell rechallenge. Specifically, as shown in FIG. 8B, 10 days after being challenged by target cells in vitro, the CAR-T cells were injected into tumor-bearing mice, 4 days after subcutaneously inoculating 1×10⁶ tumor cells, 2×10⁵ CAR-T cells as described above were injected into the mice via tail vein, in which each group included 6 mice, and each line represented one mouse. In this case, only one mouse treated with WT CAR-T cells inhibited tumor growth, while KR CAR-T cells completely eliminated tumor cells in the mice in a relatively short period of time. As shown in FIG. 8C. 21 days after being challenged by target cells in vitro, the CAR-T cells were re-injected into tumor-bearing mice. At this time. 4 day's after subcutaneously inoculating 3×10⁶ tumor cells, 1×10⁷ CAR-T cells as described above are injected into the mice via tail vein, in which each group included 4-6 mice. Under this stress model with repeated challenge of target cells for a longer period of time and increased tumor burden, WT CAR-T cells have completely lost the anti-tumor capacity; while KR CAR-T cells eventually inhibited the tumor growth despite taking a longer time. The above in-vivo experimental results fully indicated that the KR mutation prolonged the function of 41BB CAR-T cells.

In clinical practice, it is generally necessary to use the patient s limited autologous T cells to produce a large dose of CAR-T cells for reinfusion therapy, which must face the contradiction between long-term in-vitro expansion and decreased T cell function. In our experiments, KR-mutated 41BBζ CAR-T cells can still exert effective anti-tumor effects in tumor models after long-term in-vitro expansion and antigen challenge, which suggests that our design using KR mutations is expected to realize the possibility that CAR-T cells cultured in vitro for a long time can still maintain high anti-tumor efficacy in clinical production.

The foregoing descriptions are merely preferred embodiments of the present invention, and are not intended to limit the present invention in any form or substantially. It should be noted that, a person of ordinary skill in the art may make some improvements and supplements without departing from the methods of the present invention. The improvements and supplements shall fall within the protection scope of the present invention. A person skilled in the art, without departing from the spirit and scope of the present invention, may make some changes, modifications, or evolutionary equivalent changes by using the content disclosed above, all of which are equivalent embodiments of the present invention, and meanwhile, any simple change, modification, and evolution made to the above embodiments according to the technical essence of the present invention shall fall within the scope of the technical solutions of the present invention.

1. A chimeric antigen receptor, comprising:
an extracellular domain, a transmembrane domain, and an intracellular domain connected in sequence;
the extracellular domain comprises an antigen recognition region;
the intracellular domain comprises a costimulatory signaling region and a CD3ζ intracellular region connected in sequence, to form a costimulatory signaling region-CD3ζ intracellular region; and
the costimulatory signaling region-CD3ζ intracellular region is a polypeptide formed by mutation of lysine in a wild-type costimulatory signaling region-CD3ζ intracellular region into arginine.

2. The chimeric antigen receptor according to claim 1, wherein the costimulatory signaling region is selected from an intracellular region of CD27, CD28, CD134, 41BB or ICOS.

3. The chimeric antigen receptor according to claim 2, further comprising one or more of the following features:
(1) the amino acid sequence of the 41BB intracellular region is shown in SEQ ID NO: 1;
(2) the amino acid sequence of the CD28 intracellular region is shown in SEQ ID NO: 2;
(3) the amino acid sequence of the CD3ζ intracellular region is shown in SEQ ID NO: 3; and
(4) the amino acid sequence of the costimulatory signaling region-CD3ζ intracellular region is shown in SEQ ID NO: 4 or 5.

4. The chimeric antigen receptor according to claim 1, further comprising one or more of the following features:
a. the antigen recognition region is selected from a single chain antibody against a tumor surface antigen, and the tumor surface antigen is selected from one or more of CD19, CD123, CD30, BCMA, Her2, IL13Rα2 and GD2;
b. the extracellular domain further comprises a signal peptide and/or a hinge region, to form a signal peptide-antigen recognition region and hinge region connected in sequence;
c. the transmembrane domain is selected from a transmembrane region of CD4, CD8α, OX40 or H2-Kb; and
d. the amino acid sequence of the chimeric antigen receptor is shown in SEQ ID NO: 10 or SEQ ID NO: 11.

5. The chimeric antigen receptor according to claim 4, wherein the single chain antibody in feature a is selected from FMC63 or 14g2a.

6. The chimeric antigen receptor according to claim 4, wherein the signal peptide in feature b is selected from a CD8α signal peptide, and/or the hinge region is selected from a CD8α hinge region.

7. A polynucleotide sequence, wherein the polynucleotide sequence is selected from:
(1) a polynucleotide sequence encoding the chimeric antigen receptor as described in claim 1; and
(2) a complementary sequence of the polynucleotide sequence as described in (1).

8. The polynucleotide sequence according to claim 7, wherein the polynucleotide sequence is shown in SEQ ID NO: 12 or SEQ ID NO: 13.

9. A nucleic acid construct, wherein the nucleic acid construct contains the polynucleotide sequence as described in claim 7;
preferably, the nucleic acid construct is a vector; and
more preferably, the nucleic acid construct is a lentiviral vector containing a replication initiation site, 3′LTR, 5′LTR, and the polynucleotide sequence as described in claim 7.

10. A lentivirus, wherein the lentivirus contains the nucleic acid construct as described in claim 9.

11. A method for activating T cells in vitro, wherein the method comprises the operation of infecting the T cells with the lentivirus as described in claim 10.

12. A genetically modified T cell or a pharmaceutical composition containing the genetically modified T cell, wherein the cell contains the polynucleotide sequence as described in claim 7.

13. Use of the chimeric antigen receptor as described in claim 1 preparing activated T cells and/or inhibiting T cell degradation.

14. Use of the chimeric antigen receptor as described in claim 1 in any one or more of the following applications: (1) preparing tumor therapeutic drugs; (2) improving tumor killing efficiency; (3) maintaining T cell proliferation capacity; and (4) inhibiting tumor development;
preferably, the tumor is selected from one or more of leukemia or lymphoma; and
more preferably, the tumor is selected from B-cell lymphoma, mantle cell lymphoma, acute lymphocytic leukemia, chronic lymphocytic leukemia, hairy cell leukemia, and acute myeloid leukemia.

15. A genetically modified T cell or a pharmaceutical composition containing the genetically modified T cell, wherein the cell contains the nucleic acid construct as described in claim 9, or is infected with a lentivirus, or is prepared by a method;
wherein the lentivirus contains the nucleic acid construct; the method comprises the operation of infecting the T cells with the lentivirus.

16. Use of the polynucleotide sequence as described in claim 7 in preparing activated T cells and/or inhibiting T cell degradation.

17. Use of the nucleic acid construct as described in claim 9 or a lentivirus in preparing activated T cells and/or inhibiting T cell degradation;
wherein the lentivirus contains the nucleic acid construct.

18. Use of the polynucleotide sequence as described in claim 7 in any one or more of the following applications: (1) preparing tumor therapeutic drugs; (2) improving tumor killing efficiency; (3) maintaining T cell proliferation capacity; and (4) inhibiting tumor development;
preferably, the tumor is selected from one or more of leukemia or lymphoma; and more preferably, the tumor is selected from B-cell lymphoma, mantle cell lymphoma, acute lymphocytic leukemia, chronic lymphocytic leukemia, hairy cell leukemia, and acute myeloid leukemia.

19. Use of the nucleic acid construct as described in claim 9 or a lentivirus in any one or more of the following applications: (1) preparing tumor therapeutic drugs; (2) improving tumor killing efficiency; (3) maintaining T cell proliferation capacity; and (4) inhibiting tumor development;
preferably, the tumor is selected from one or more of leukemia or lymphoma; and
more preferably, the tumor is selected from B-cell lymphoma, mantle cell lymphoma, acute lymphocytic leukemia, chronic lymphocytic leukemia, hairy cell leukemia, and acute myeloid leukemia;
wherein the lentivirus contains the nucleic acid construct.

20. Use of the genetically modified T cell as described in claim 12 in any one or more of the following applications: (1) preparing tumor therapeutic drugs; (2) improving tumor killing efficiency; (3) maintaining T cell proliferation capacity; and (4) inhibiting tumor development;
preferably, the tumor is selected from one or more of leukemia or lymphoma; and more preferably, the tumor is selected from B-cell lymphoma, mantle cell lymphoma, acute lymphocytic leukemia, chronic lymphocytic leukemia, hairy cell leukemia, and acute myeloid leukemia.
